(19) United States
(12) Patent Application Publication
(10) Pub. No.: US 2011/0269755 A1
(43) Pub. Date: Nov. 3, 2011

(54) COMPOSITIONS AND METHODS FOR TREATING VASCULAR, AUTOIMMUNE, AND INFLAMMATORY DISEASES

(75) Inventors: Michael HAYDEN, Vancouver (CA); Noel Hall, Victoria (CA)

(73) Assignee: Aspreva International Ltd., Victoria (CA)

(21) Appl. No.: 12/964,722

(22) Filed: Dec. 9, 2010

Related U.S. Application Data
(63) Continuation of application No. 12/377,384, now abandoned.
(60) Provisional application No. 60/838,231, filed on Aug. 16, 2006.

(51) Int. Cl. A61K 31/5377; A61K 31/351; A61K 31/40; A61P 1/16; A61K 31/405; A61P 3/06; A61P 9/10; A61P 3/10; A61K 31/343; A61K 31/505

(52) U.S. Cl. 514/233.5; 514/470; 514/460; 514/423; 514/275; 514/419

(57) ABSTRACT

The disclosure provides compositions and methods for the treatment of vascular, autoimmune, and inflammatory diseases using a combination of an inosine monophosphate dehydrogenase (IMPDH) inhibitor and a HMG CoA reductase inhibitor.

### 1. TECHNICAL FIELD

The present disclosure relates to compositions and methods for treating vascular, autoimmune, and inflammatory diseases.

### 2. BACKGROUND

Inosine monophosphate dehydrogenase (IMPDH) is the rate-limiting enzyme in the de novo biosynthesis of guanosine nucleotides in mammals. Both T- and B-lymphocytes rely exclusively on de novo guanosine nucleotide synthesis as they are deficient in salvage pathways.

Mycophenolic acid (“MPA”) is a potent inhibitor of IMPDH that has gained widespread acceptance as an immunosuppressant, particularly in the prophylactic treatment of organ rejection in patients receiving allogenic renal, cardiac or hepatic transplants. MPA treatment in the form of the 2-morpholinoethyl ester prodrug mycophenolate mofetil (“MMF”; structure illustrated below) is marketed in the US for these indications by Hoffman LaRoche under the tradename CellCept®:

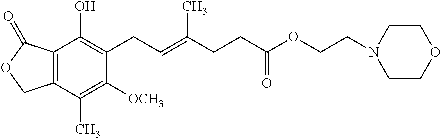

CellCept® is currently available in capsule (250 mg), tablet (250 mg and 500 mg), oral suspension (200 mg/ml when constituted) and intravenous (6 mg/ml in 5% dextrose when reconstituted) dosage forms. Following oral or intravenous administration, the MMF is rapidly and completely metabolized to the active metabolite MPA (see, e.g., Physicians Desk Reference, 2005 Ed., pp. 2855; “PDR”).

A delayed-release, enterically coated tablet formulation of the sodium salt of mycophenolic acid (mycophenolate sodium) is marketed in the U.S. by Novartis AG under the tradename Myfortic®. Each tablet contains either 180 mg or 360 mg of mycophenolate sodium. According to the 2005 Edition of the PDR, Myfortic® is currently approved for the prophylactic treatment of organ rejection in patients receiving allogenic renal transplants.

Lipid lowering agents are another class of therapeutic agent which has achieved widespread commercial use. Major components of this class include the statins, nicotinic acid (niacin), and fibric acid derivatives. The statins are inhibitors of 3-hydroxy-3-methylglutaryl coenzyme A (HMG-CoA) reductase. An exemplary statin is atorvastatin (Lipitor®), which is a pharmaceutical salt preparation and has the following structural formula:

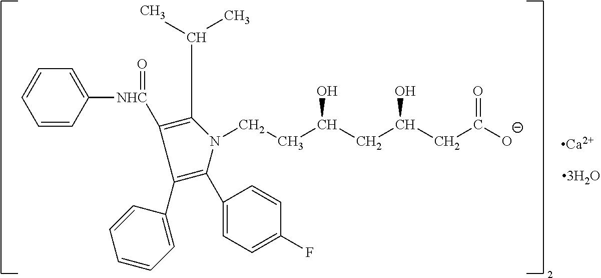

Clinical and laboratory evidence indicate that statins have pleiotropic effects in addition to lipid-lowering activity. The mechanism of statins' varied effects remains poorly understood, but various hypotheses have been cited (Ehrenstein et al., 2005, N. Engl J Med. 352:1-3; Barilla-LaBarca et al., 2003, Curr Opin Rheumatol. 15(1):55-60; Carroll, M. C., 2004, Nat Rev Immunol. 4(10):825-31).

A need exists to identify improved therapeutic compositions that can be used for the treatment of vascular, autoimmune and inflammatory diseases. The instant disclosure provides such therapeutic compositions and methods, relying on a novel understanding of these compositions and their combined utility in treating certain vascular, autoimmune and inflammatory disease processes.

### 3. SUMMARY

In one aspect, the present disclosure provides methods for treating selected vascular, autoimmune and inflammatory diseases in a subject by adjunctively administering to the subject an IMPDH inhibitor and an HMG-CoA reductase inhibitor (including their corresponding salts, hydrates, and solvates).

The IMPDH inhibitor can be any compound that inhibits the activity of IMPDH, or a prodrug of such an IMPDH-inhibitory compound (i.e., a compound that metabolizes under conditions of use to a compound that inhibits the activity of IMPDH). Such IMPDH inhibitory compounds and prodrugs are well-known, and include, by way of example and not limitation, inhibitors 3-(1-deoxy-beta-D-ribofuranosyl)benzamide (Jayaram et al., 1992, Biochem Biophys Res Commun. 186(3):1600-6), mizoribine, 5-beta-D-ribofuranosylselenophene-3-carboxamide (Franchetti et al., 1997, J Med Chem. 40(11):1731-7), N-[2-[2-[[3-methoxy-4-(5-oxazolyl)phenyl]amino]-5-oxazolyl]phenyl]-N-methyl-4-morpholineacetamide (Dhar et al., 2002, J Med Chem. 45(11):2127-30), and mycophenolic acid and their various corresponding salts, hydrates, solvates and esters. Other IMPDH inhibitors include compounds disclosed in U.S. Pat. Nos. 5,807.876; 5,932,600; 6,054,472; 6,344,465; 6,420,403; 6,518,291; 6,541,496; 6,596,747; 6,617,323; and 6,624,184. In some embodiments, the IMPDH inhibitory compound administered is mycophenolic acid and/or a salt, hydrate, solvate and/or ester thereof. In a specific embodiment, the compound administered is selected from a salt of mycophenolic acid, such as, for example, mycophenolate sodium, and an ester of mycophenolic acid (MPA), such as, for example, mycophenolate mofetil (MMF).

The HMG CoA reductase inhibitor can be any compound that inhibits the activity of HMG CoA reductase, or a prodrug of such a HMG CoA reductase inhibitory compound. A useful class of HMG CoA reductase inhibitors is statins, which are generally prescribed for treating a hypercholesteremic condition. Exemplary HMG CoA reductase inhibitors include, among others, mevastatin, lovastatin, simvastatin, pravastatin, fluvastatin, atorvastatin, pitavastatin, and rosuvastatin.

The IMPDH and HMG-CoA reductase inhibitor compounds can be administered simultaneously, either as separate dosage forms or as a single combination dosage form. Alternatively, they may be administered at different times.

The combination therapy may be practiced in patients suffering from a vascular, autoimmune and/or inflammatory disease, or a condition associated with such diseases. In some embodiments, the combination of drugs can be administered prophylactically to patients that do not currently suffer from such a disease or condition. Thus, in some embodiments, the prophylactic therapy can be practiced in patients that are at risk of developing a vascular, autoimmune and/or inflammatory disease or a condition associated with a vascular, autoimmune and/or inflammatory disease.

In some embodiments, the condition treated is associated with an autoimmune disease, such as systemic lupus erythematosus, rheumatoid arthritis, and diabetes mellitus, and includes conditions such as atherosclerosis, cardiovascular disease, and other vascular diseases, which occur at higher frequency in patients with the particular autoimmune disorder. In other embodiments, the condition associated with the autoimmune disease is an inflammatory reaction, which typically accompanies many different autoimmune reactions. In various embodiments, the combination of drugs may be administered at doses effective to treat or reduce the risk of developing the associated condition regardless of the effectiveness of the drugs on the underlying disease.

The amounts of the IMPDH and HMG CoA reductase inhibitors administered can be the standard dosages typically administered for their approved indications (as specified in The 2005 edition of The Physician's Desk Reference; “PDR”), or alternatively, the amounts administered for either or both compounds can be selectively varied. In some embodiments, it may be desirable to administer an amount of IMPDH inhibitor that provides therapeutic or prophylactic benefit for the indications described herein but that is less than the amount typically administered for the treatment or prophylaxis of allograft transplant rejection. In this way, therapeutic and/or prophylactic benefit may be achieved without compromising the immune system of the subject. Thus, in some embodiments, the daily amount of IMPDH inhibitor MMF administered can correspond to a dose equivalent to a human dose of about 5 to about 50 mg/day, above about 50 to less than about 100 mg/day, about 100 to less than about 250 mg/day, about 250 to less than about 500 mg/day, or about 0.5 to about 1.0 g/day.

The combination therapy can utilize a single compound of each class, for example a single IMPDH inhibitor and a single HMG CoA reductase inhibitor, or multiple compounds from each class, for example, a single IMPDH inhibitor and two different HMG CoA reductase inhibitors, two different IMPDH inhibitors and a single HMG CoA reductase inhibitor, two different IMPDH inhibitors and two different HMG CoA reductase inhibitors, etc.

In another aspect, the present disclosure provides pharmaceutical compositions comprising a HMG CoA reductase inhibitor and an IMPDH inhibitor (including salts, hydrates and/or solvates of such compounds) and one or more pharmaceutically acceptable carriers, excipients and/or diluents. In some embodiments, the amounts of the compounds included in the composition are specifically suited to provide therapeutic and/or prophylactic benefit in the methods described herein. Accordingly, in some embodiments, the compositions comprise unit dosage amounts or fractional unit dosage amounts of the IMPDH inhibitor(s) that are tailored to administer less than the standard dosages.

In another aspect, the present disclosure provides methods of treating and/or preventing restenosis, which typically arises from vascular reconstructive procedures such as the use of a stent to open clogged arteries. The methods generally comprise adjunctively administering to a subject an effective amount of an IMPDH inhibitor and a HMG CoA reductase inhibitor. The HMG CoA reductase inhibitor and IMPDH inhibitor can be administered to the subject via systemic routes of administration, or provided in the stent itself for local administration, such as in a polymer coating on the stent. Polymer coatings that allow slow or quick release of the compounds can be used.

In another aspect, the present disclosure provides kits useful for practicing the various methods described herein. In some embodiments, the kits comprise an IMPDH inhibitor and a HMG CoA reductase inhibitor in formulations suitable for administration to subjects. The compounds can be in separate containers, or provided as compositions, either as solid dosages or liquid formulations. The kits may further comprise devices for their administration and/or instructions for proper dosing.

### 4. BRIEF DESCRIPTION OF THE FIGURES

FIG. 1 shows the effect of mycophenolate mofetil given at dosages of 6, 15, 30 and 100 mg/kg/day in reducing atherosclerosis (AS), as assessed by percentage of aortic sinus occluded by plaque, in male Ldlr−/− mice. A dose of 6 mg/kg/day of MMF, a dosage equivalent to approximately 30 milligrams per day in a 60 kg human, reduced AS by 18%, whereas 15 mg/kg/day of MMF, a dosage equivalent to approximately 75 milligrams per day in a 60 kg human, reduced AS by 30%. A dose of 30 mg/kg/day of MMF, a dosage equivalent to approximately 150 milligrams per day in a 60 kg human also reduced AS by 28%. The highest dose tested, 100 mg/kg/day of MMF, a dosage equivalent to approximately 500 milligrams per day in a 60 kg human, which is lower than the standard dose employed for transplantation purposes, reduced AS by 64%. The response was dose-dependent (statistical analysis shown on lower left). Quantification was performed by a single, trained observer blinded to treatment group.

FIG. 2 shows the effect of various dosages of MMF on serum triglyceride (Trigs) levels in male Ldlr−/− mice.

FIG. 3 shows the effect of various dosages of MMF in reducing serum phospholipid (PPL) levels in male Ldlr−/− mice.

FIG. 4 shows the effect of MMF on total serum cholesterol and high-density lipoprotein cholesterol levels in male Ldlr−/− mice at dosages of 15 mg/kg/day and 100 mg/kg/day.

FIG. 5 shows the correspondence between serum levels of MPA and various doses of MMF administered to Ldlr−/− mice. The “therapeutic window” of serum MPA levels is approximately 1.0 to about 3.5 mcg/mL.

FIG. 6A shows the correlation between serum levels of MPA and the reduction in atherosclerotic plaques. The therapeutic window is highlighted. FIG. 6B shows a linear regression analysis of the data in FIG. 6A, indicating a statistically significant decreasing trend in aortic root lesions as a function of serum MPA levels.

FIG. 7 shows the effect of administering HMG CoA reductase inhibitor (i.e., simvastatin) alone or a combination of HMG CoA reductase inhibitor and MMF to Ldlr−/− mice on the level of atherosclerotic plaques. Simvastatin was given at 90/mg/kg/day while MMF was given at 30 mg/kg/day. Animals were 14.5 weeks old at the beginning of the treatment, which lasted for 8 weeks.

FIG. 8 shows the reduction in atherosclerotic plaques in male Ldlr−/− mice treated with MMF alone at a dosage of 30 mg/kg/day (data of FIG. 1), simvastatin alone at 90 mg/kg/day, and a combination of MMF and simvastatin (data of FIG. 7).

FIG. 9 shows the percentages of large vessels branching off the aorta that showed plaque in male Ldlr−/− mice treated with a combination of MMF and simvastatin. MMF was administered at the various dosages shown while simvastatin was kept constant at 90 mg/kg/day.

FIG. 10 shows the serum triglyceride levels in male Ldlr−/− mice treated with simvastatin alone (90 mg/kg/day) or a combination of simvastatin (90 mg/kg/day) and MMF (30 mg/kg/day).

FIG. 11 shows the phospholipids levels in male Ldlr−/− mice treated with simvastatin alone (90 mg/kg/day) or a combination of simvastatin (90 mg/kg/day) and MMF (30 mg/kg/day).

FIG. 12 shows the total serum cholesterol levels in male Ldlr−/− mice treated with simvastatin alone (90 mg/kg/day) or a combination of simvastatin (90 mg/kg/day) and MMF (30 mg/kg/day).

FIG. 13 shows a study of female Ldlr−/− treated with simvastatin alone (90 mg/kg/day) or a combination of simvastatin (90 mg/kg/day) and MMF (15 mg/kg/day). A lower number of aortic lesions was observed for the group treated with the combination as compared to monotherapy with simvastatin. Animals were 8 weeks of age, and treatment was carried out for 8 weeks.

FIG. 14 shows the level of natural killer T cells (NKT cells) in the spleen of female Ldlr−/− mice treated with simvastatin alone (90 mg/kg/day) or a combination of simvastatin (90 mg/kg/day) and MMF (15 mg/kg/day).

### 5. DETAILED DESCRIPTION

The present disclosure provides methods and compositions for treating vascular, autoimmune and inflammatory diseases in a subject. The methods comprise adjunctively administering to the subject an effective amount of a combination of an IMPDH inhibitor and a HMG-CoA reductase inhibitor. This combination therapy may provide treatment for indications not previously achieved by either drug separately.

From studies of patients afflicted with systemic lupus erythematosus (SLE), an autoimmune disease with an associated inflammatory component, the inventors recognize that the risk of myocardial infarction is increased by as much as 9-fold in patients with SLE, even after adjustment for cardiovascular disease (CVD) risk factors such as hypertension and high cholesterol (Esdaile et al., 2001, Arth. & Rheum. 44(10):2331-2337). Unstable, rupture prone plaques, which are thought to be responsible for incidences of myocardial infarction and other ischemic events, have identifiable features, including numerous inflammatory cells. These unstable plaques are characterized by active inflammation that may overwhelm the plaque's capacity for repair (Ross R., 1999, Atherosclerosis—an inflammatory disease,” N Engl J Med. 340:115-126). Macrophages and T cells are abundant in the regions of plaque rupture, while smooth muscle cells are few. Stable plaques, conversely, contain few inflammatory cells and have abundant smooth muscle cells. Thus, a defective inflammatory response involved in SLE may be responsible for both the underlying disease and the cardiovascular outcomes of the disease.

On the other hand, clinical and genetic studies in humans and animal models indicate a crucial protective role for the complement system in systemic lupus erythematosus (SLE). This presents a paradox because the complement system is considered to be an important mediator of the inflammation that is observed in patients with SLE. One current view is that complement provides protection by facilitating the rapid removal of apoptotic debris to circumvent an autoimmune response. (Barilla-LaBarca et al., 2003, Curr Opin Rheumatol. 15(1):55-60) In an alternative model, complement, together with other components of the innate immune system, participates in the ‘presentation’ of SLE-inducing self-antigens to developing B cells. In this way, the complement system and innate immunity may protect against responses to SLE (self) antigens by enhancing the elimination of self-reactive lymphocytes. (Carroll, M C., 2004, Nat Rev Immunol. 4(10):825-31).

Because the inflammatory component and the self reactive lymphocytes prominent in SLE may be responsible for the adverse physiological effects seen in patients afflicted with SLE, immunosuppressive therapy using cytotoxic agents may provide a therapeutic benefit to such patients by reducing the inflammatory reaction and attenuating the activity of self-reactive lymphocytes. A class of useful immunosuppressives for this purpose is IMPDH inhibitors, such as mycophenolic acid and its prodrug form mycophenolate mofetil, which targets the enzyme catalyzing the rate-limiting step in the de novo biosynthesis of guanine nucleotides from inosine. Since T and B-lymphocytes rely almost exclusively on the de novo pathway of purine synthesis, IMPDH inhibitors specifically target the proliferation of T and B cells, thereby inhibiting production of antibodies and generation of cytotoxic T lymphocytes. This degree of specificity is a desirable characteristic for immunosuppressive therapy when the therapeutic mechanism is cytotoxicity. In addition, it has been noted that in some disease states, such as atherosclerosis, T-regulatory cells elaborate numerous cytokines that influence the recruitment of monocyte macrophages into tissue macrophages. Thus, therapies targeted at individual cytokines would be expected to show diminished efficacy when compared to a therapy that targets the T-cell as a complete unit (Freeman M, “Type II Diabetes and Atherosclerosis”—Oral Presentation at the Keystone Meeting on Adipogenesis, Obesity and Inflammation, Vancouver, B.C., Jan. 25, 2006).

However, in developing effective therapeutic treatments, use of a combination of drugs (i.e., drug cocktails) that act by different mechanisms but which affect the same underlying cause of the disorder may be more effective than use of a single therapeutic agent. This “multi-hit” approach to therapeutics may be more beneficial for several reasons: (1) less variability in the response of the treated population to several therapeutic agents compared to a single therapeutic agent, and (2) stricter modulation of the physiological processes responsible for the disease by targeting multiple biochemical pathways that impinge upon the disease process.

Statins, a class of HMG CoA reductase inhibitors, are generally prescribed to treat hyperlipidemic conditions characterized by elevated cholesterol levels. Statins, however, have pleiotropic effects on the vasculature and the immune system, independent of statins' ability to modulate serum cholesterol.

Statins can directly upregulate endothelial nitric oxide synthase (eNOS) expression in vitro under cholesterol clamped conditions (Laufs et al., 1998, “Upregulation of endothelial nitric oxide synthase by HMG CoA reductase inhibitors,” Circulation 97:1129-1135). Both simvastatin and lovastatin upregulate eNOS expression almost fourfold, and completely prevent its downregulation by oxidized LDL. The upregulation of eNOS is reversed by the addition of mevalonate. In addition, a significant increase in endothelium-dependent vasodilation in patients with moderate hypercholesterolemia is observed after 4 weeks of treatment with simvastatin (O'Driscoll et al., 1997, “Simvastatin, an HMG-coenzyme A reductase inhibitor, improves endothelial function within 1 month,” Circulation 95:1126-1131). The neuroprotective effect of statins is absent in eNOS deficient mice, suggesting that enhanced eNOS activity by statins is a main mechanism by which HMG CoA reductase inhibitors protect against cerebral injury (Endres et al., 1998, “Stroke protection by 3-hydroxy-3-methylglutaryl (HMG)-CoA reductase inhibitors mediated by endothelial nitric oxide synthase,” Proc Natl Acad Sci USA 95:8880-8885).

Furthermore, the statin fluvastatin appears to decrease MMP-1 expression in human vascular endothelial cells in a time- and dose-dependent manner (Ikeda et al., 2000, “Fluvastatin inhibits matrix metalloproteinase-1 expression in human vascular endothelial cells,” Hypertension 36:325-329). This effect is also seen with lovastatin and again is completely blocked by coincubation with mevalonate. The concentration of fluvastatin required to reduce MMP-1 expression is similar to that seen in clinical practice.

Pravastatin has been shown to cause changes in the composition of atheromatous plaque independent of its cholesterol lowering effect. Pravastatin-treated monkeys have enhanced vasodilator function and favorable changes in the composition of atheromatous plaque compared with control animals with similar changes in lipid profile caused by diet alone (Williams et al., 1998, “Pravastatin has cholesterol-lowering independent effects on the artery wall of atherosclerotic monkeys,” J Am Coll Cardiol 31:684-691). The pravastatin-treated monkeys had fewer macrophages in the intima and media, less calcification and less neovascularization in the intima. Pravastatin may thus serve to stabilize vulnerable plaques by promoting regression of fragile, rupture prone microvessels in the intima.

Numerous studies also suggest important effects of statins on macrophage and T-Cell function. Macrophages are capable of degrading the extracellular matrix and, by secreting matrix metalloproteinase (MMP), may weaken the fibrous cap and thus predispose an atheromatous plaque to rupture. Fluvastatin and simvastatin have been shown to inhibit MMP-9 (gelatinase B) activity and secretion by macrophages (Bellosta et al., 1998, “HMG-CoA reductase inhibitors reduce MMP-9 secretion by macrophages,” Arterioscler Thromb Vasc Biol. 18:1671-1678). This effect is reversed by the addition of mevalonate, suggesting that it is mediated by HMG CoA reductase inhibition.

Hypercholesterolemic rats treated with fluvastatin have attenuated leukocyte adherence responses to platelet activation factor and leukotriene B4 (Kimura M. et al., 1997, “Effects of fluvastatin on leukocyte-endothelial cell adhesion in hypercholesterolemic rats,” Arterioscler Thromb Vasc Biol 17:1521-1526). Statins inhibit the expression of CD-11b on the cell surface, thus reducing the adhesiveness of macrophages to the vascular endothelium (Weber et al., 1997, “ HMG-CoA reductase inhibitors decrease CD11b expression and CD11b-dependent adhesion of monocytes to endothelium and reduce increased adhesiveness of monocytes isolated from patients with hypercholesterolemia,” J Am Coll Cardiol 30:1212-1217). Atorvastatin reduces monocyte chemo-attractant protein-1 levels in the intima and media in hypercholesterolemic rabbits (Bustos et al., 1998, “HMG-CoA reductase inhibition by atorvastatin reduces neointimal inflammation in a rabbit model of atherosclerosis,” J Am Coll Cardiol 32:2057-2064). This decrease in monocyte chemo-attractant protein-1 is related to a reduction in nuclear factor κB activation, a transcription factor involved in the induction of monocyte chemo-attractant protein-1 and other proinflammatory cytokines such as IL-1β and tumor necrosis factor-α (TNF-α).

Statins also cause a decrease in macrophage expression of soluble intercellular adhesion molecule-1 and lipopolysaccharide-induced secretion of IL-6 and TNF-α by monocytes and macrophages (Niwa et al., 1996, “Inhibitory effect of fluvastatin, an HMG-CoA reductase inhibitor, on the expression of adhesion molecules on human monocyte cell line,” Int J Immunopharmacol 18:669-675; Ikeda et al., 1999, “Statins and monocytes,” Lancet 353:2070; and Rosenson et al., 1999, “Inhibition of proinflammatory cytokine production by pravastatin,” Lancet 353:983-984). Simvastatin therapy for 8 weeks reduces monocyte expression of TNF-α and IL-1β by 49 and 35%, respectively (Ferro et al., 2000, “Simvastatin inhibits the monocyte expression of proinflammatory cytokines in patients with hypercholesterolemia,” J Am Coll Cardiol 36:427-431); this is intriguing data because elevated plasma levels of both soluble intercellular adhesion molecule-1 and IL-6 have been shown to predict risk for myocardial infarction (Ridker et al., 2000, “Plasma concentration of interleukin-6 and the risk of future myocardial infarction among apparently healthy men,” Circulation 101:1767-1772; Ridker et al., 1998, “Plasma concentration of soluble intercellular adhesion molecule 1 and risks of future myocardial infarction in apparently healthy men,” Lancet 351:88-92). A recent analysis from the Cholesterol and Recurrent Events (CARE) trial showed that plasma concentrations of TNF-α are also persistently elevated among postmyocardial infarction patients at increased risk for coronary events (Ridker et al., 2000, “Elevation of tumor necrosis factor-alpha and increased risk of recurrent coronary events after myocardial infarction,” Circulation 101:2149-2153).

The anti-inflammatory activity of statins may also be explained based on the role of oxidized LDL in the atherogenic pathway. The uptake of oxidized LDL by macrophages generates lipid rich foam cells. Oxidized LDL causes monocyte tissue factor expression, and the proliferation and apoptosis of smooth muscle cells (Bjorkerud et al., 1996, “Contrary effects of lightly and strongly oxidized LDL with potent promotion of growth versus apoptosis on arterial smooth muscle cells, macrophages, and fibroblasts,” Arterioscler Thromb Vasc Biol 16:416-424; Broze, G J, 1992, “The role of tissue factor pathway inhibitor in a revised coagulation cascade,” Semin Hematol 29:159-169). Oxidized LDL also inhibits nitric oxide synthase activity and hence impairs endothelium-dependent vasodilation (Laufs et al., 1998, “Upregulation of endothelial nitric oxide synthase by HMG CoA reductase inhibitors,” Circulation 97:1129-1135). Statins reduce the susceptibility of LDL to oxidation by a variety of mechanisms. Statins reduce the cholesterol content of lipoproteins through their hypocholesterolemic effects, and thus lower the amount of substrate available for oxidation (Hoffman et al., 1992, “Hypolipidemic drugs reduce lipoprotein susceptibility to undergo lipid peroxidation: in vitro and ex vivo studies,” Atherosclerosis 93:105-113). Simvastatin also reduces macrophage superoxide formation, thereby decreasing cell oxygen production (Giroux et al., 1993, “Simvastatin inhibits the oxidation of low-density lipoproteins by activated human monocyte-derived macrophages,” Biochim Biophys Acta 1165:335-338). Fluvastatin and lovastatin bind to phospholipid on the surface of LDL and thus prevent diffusion into the lipoprotein core of free radicals generated under oxidative stress (Aviram et al., 1998, “Interactions of platelets, macrophages, and lipoproteins in hypercholesterolemia: antiatherogenic effects of HMG-CoA reductase inhibitor therapy,” J Cardiovasc Pharmacol 31:39-45). Atorvastatin and fluvastatin have also been shown to have direct antioxidant potential (Aviram et al., 1998, “Atorvastatin and gemfibrozil metabolites, but not the parent drugs, are potent antioxidants against lipoprotein oxidation,” Atherosclerosis 138:271-280; Suzumura et al., 1999, “Protective effect of fluvastatin sodium (XU-62-320), a 3-hydroxy-3-methylglutaryl coenzyme A (HMG-CoA) reductase inhibitor, on oxidative modification of human low-density lipoprotein in vitro,” Biochem Pharmacol 57:697-703).

Pravastatin therapy is associated with a reduction in the number of episodes of rejection following cardiac transplantation. The inhibition of natural killer T cell activity by pravastatin may explain, in part, this beneficial effect (Kobashigawa et al., 1995, N Engl J Med. 333(10):621-7). Although transplant vasculopathy is an entity distinct from atherosclerotic disease, similar inflammatory mediators may contribute to plaque instability.

There is also an indication that statins may be beneficial for multiple sclerosis and other Th1-mediated autoimmune disease. (Yousseff et al. 2002, Nature. 420(6911):78-84). This conclusion was based on the finding of direct effects of atorvastatin (Lipitor®) on differentiation of Th0 cells into Th2 cells in the mouse model of chronic and relapsing experimental autoimmune encephalomyelitis (EAE). Atorvastatin can induce STAT6 phosphorylation and secretion of Th2 cytokines (interleukin (IL)-4, IL-5 and IL-10) and transforming growth factor (TGF)-beta. Conversely, STAT4 phosphorylation was inhibited and secretion of Th1 cytokines (IL-2, IL-12, interferon (IFN)-gamma and tumor necrosis factor (TNF)-alpha) was suppressed. Thus, atorvastatin has pleiotropic immunomodulatory effects involving both APC and T-cell compartments.

The pleiotropic biological properties of statins may be attributable to their effect on the synthesis of intermediates used as lipid attachments for the modification and membrane localization of proteins. Farnesyl and geranylgeranyl groups are found on a variety of proteins, including heterotrimeric G proteins, nuclear lamins, and small GTP-binding proteins, such as ras, rho, rab rac, ral and rap. Inhibiting lipid attachment results in protein mislocalization in the cytoplasm, thereby disrupting proper protein function. The pleiotropic effects of statins may arise from the critical role played by many of these lipidated proteins (e.g., ras and rho) in signal transduction pathways.

Given the pleiotropic effects of statins and its posited mechanism of action, treatment with a combination of a HMG CoA reductase inhibitor and an IMPDH inhibitor may provide a greater therapeutic benefit in patients afflicted with certain vascular diseases, since these agents appear to work via different mechanisms of action. It is indicated in the studies described herein that treatment with the combination of the compounds in animal model systems of atherosclerosis reduces the number of atherosclerotic plaques below those observed when the compounds are used alone (i.e., as monotherapy). Along with the decrease in the indicator of atherosclerosis are concomitant decreases in serum cholesterol, serum triglyceride, and serum phospholipids to levels that are lower than what is observed when the individual compounds are used alone. Accordingly, the present disclosure provides a method of treating vascular diseases, such as atherosclerosis, with a combination of an IMPDH inhibitor and a HMG CoA reductase inhibitor. In other embodiments, the combination can also be used to treat autoimmune and inflammatory diseases.

As used herein, an “HMG-CoA reductase inhibitor” includes any compound that inhibits or reduces the biological activity of 3-hydroxy-3-methylglutaryl coenzyme A (HMG-CoA) reductase. One important class of HMG CoA reductase inhibitors is statins, various embodiments of which are known in the art. Atorvastatin, and derivatives thereof, are described in U.S. Pat. No. 5,273,995 and EP 409281 and are available commercially under the tradenames Lipitor®, Sortis®, Torvast®, Totalip®, and Xarator®. Cerivastatin is described in U.S. Pat. Nos. 5,006,530 and 5,177,080, and EP 325130, and is available under the tradenames Rivastatin®, Baycol®, and Lipobay®. Although the levels of cerivastatin prescribed for hyperlipidemia has resulted in toxic side effects, lower non-toxic levels may be appropriate for treatments described herein. Lovastatin and derivatives thereof are described in U.S. Pat. No. 4,231,938 and are available under the tradenames Altocar®, Lovalip®, Mevacor®, Mevinacor®, Nevlor®, and Sivlor®. Pitavastatin and derivatives thereof are described in EP65835 and U.S. Pat. No. 6,162,798, and are available under the tradenames Itabastatin®, Livalo®, Nisvastatin®, Itavastatin®, and Zomaril®. Pravastatin and derivatives thereof are described in U.S. Pat. No. 4,346,227 and DE 3122499 and are available under the tradenames Elisor®, Lipostat®, Liprevil®, Mevalotin®, Oliprevin®, Pravachol®, Pravasin®, Selectin®, and Vasten®. Rosuvastatin and derivatives thereof are described in U.S. Pat. Nos. 5,128,366; 5,260,440; and 6,589,959; and patent EP 521471. Simvastatin and derivatives thereof are described in U.S. Pat. No. 4,444,784 and EP 33538 and are available under the tradenames Denan®, Liponorm®, Simovil®, Sinvacor®, Sivastin®, Zocor®, and Zocord®. Fluvastatin and derivatives thereof are described in U.S. Pat. No. 4,739,073 and WO 84/02131 and are available under the tradenames Fluindostatin®, XU 62-320®, Lescol®, Lipaxan® and Primexin®. Mevastatin is described in, among others, Fears et al., 1980, Atherosclerosis 35(4):439-49, and is also known as compactin. The statins may be used individually, or as compatible mixtures to enhance efficacy and/or reduce toxicity of the HMG CoA reductase inhibitors.

In some embodiments, the combination therapy may also include other lipid lowering drugs, such as fibric acid derivatives. Exemplary fibric acid derivatives include, among others, clofibrate, colestipol, and gemfibrozil. Clofibrate is described in U.S. Pat. No. 3,262,850 and GB 860303 and is available under the tradenames Amotril®, Anparton®, Apolan®, Artevil®, Claripex®, Normet®, Regelen®, Serotinex®, and Xyduril®. Colestipol and derivatives thereof are described in U.S. Pat. Nos. 3,692,895 and 3,803,237, and patents DE 1927336 and DE 2053585. Gemfibrozil and derivatives thereof are described in U.S. Pat. Nos. 3,674,836 and 4,126,637, and patent DEL 1925423 and are available under the tradenames Decrelip®, Genlip®, Gevilon®, Lipozid®, and Lopid®.

As used herein, “IMPDH inhibitory compound” or “IMPDH inhibitor” refers to any compound that inhibits or reduces the activity of inosine monophosphate dehydrogenase (IMPDH), the rate-limiting enzyme in the de novo biosynthesis of guanosine nucleotides. Also included within the definition are prodrugs of such IMPDH inhibitory compounds (for example, esters of such compounds) that metabolize under their conditions of use to an active metabolite that is an IMPDH inhibitory compound.

Significant members of the class of IMPDH inhibitory compounds are mycophenolic acid (“MPA”) and its corresponding salts, hydrates, solvates and esters, and mycophenolate mofetil (“MMF”) and its corresponding salts, hydrates and solvates. MMF is the 2-morpholinoethyl ester prodrug of MPA. When administered orally or intravenously, MMF rapidly and completely metabolizes to MPA. MPA is a selective, non-competitive and reversible inhibitor of IMPDH (in particular the type II isoform) and strongly inhibits both T- and B lymphocyte proliferation. MMF has been used in the prevention of acute and chronic allograft rejection since the mid 1990s under the trade name CellCept® (F. Hoffman-La Roche, AG). An enterically coated formulation of the sodium salt of MPA (mycophenolate sodium) has been approved for prophylaxis of rejection in allogenic renal transplants under the tradename Myfortic® (Novartis AG). Specific embodiments of salts and analogs of MMF, as well as methods of making the salts and analogs, are described in U.S. Pat. Nos. 4,686,234; 4,725,622; 4,727,069; 4,748,173; 4,753,935; 4,786,637; 4,808,592; 4,861,776; 4,868,153; 4,948,793; 4,952,579; 4,959,387; and 4,922,467; the disclosures of which are incorporated herein by reference.

Other IMPDH inhibitors are known and include compounds disclosed in U.S. Pat. Nos. 5,807,876; 5,932,600; 6,054,472; 6,344,465; 6,420,403; 6,518,291; 6,541,496; 6,596,747; 6,617,323; and 6,624,184; the disclosures of which are incorporated herein by reference.

In various embodiments, disease areas where the disclosed combination therapy is expected to be applicable include vascular, autoimmune, and/or inflammatory diseases, or conditions associated with such diseases. As such, in some embodiments, the combination therapy can be used to treat autoimmune diseases that include, by way of example and not limitation, systemic lupus erythematosus (SLE), multiple sclerosis (MS), diabetes mellitus, and rheumatoid arthritis (RA). In some embodiments, the associated condition is an inflammatory condition associated with the autoimmune disease. For example, chronic inflammation accompanies many forms of autoimmune disease, such as rheumatoid arthritis, systemic lupus, and diabetes mellitus. The inflammatory cascade activated by the autoimmune reaction can exacerbate the damage caused by the autoimmune activity. In some cases, a patient with an underlying autoimmune disease may not display clinical signs of an inflammatory reaction, but have levels of biochemical markers indicative of inflammatory reactions (e.g., inflammatory cytokine levels). As such, the compositions and methods herein can be used to treat or reduce the risk of such inflammatory conditions associated with autoimmune disease.

In some embodiments, the combination therapy can be used to treat inflammatory diseases that include, by way of example and not limitation, Crohn's disease, ulcerative colitis, pelvic inflammation, and vasculitis. An exemplary inflammatory disease treatable with the compositions is vasculitis, a vascular inflammatory disease arising from inflammation of the blood vessel system, which includes the veins, arteries, and capillaries. Vasculitis may affect blood vessels of any type, size, or location, and therefore can cause dysfunction in any organ system, including the central and peripheral nervous systems.

In some embodiments, the compositions and methods can be used to treat vascular diseases, which include, by way of example and not limitation, cardiovascular disease (CVD), arteriosclerosis (e.g., atherosclerosis), coronary artery disease (CAD), coronary heart disease (CHD), cerebrovascular disease, and peripheral vascular disease. As used herein, “vascular disease” refers to the constellation of disorders affecting the function of arteries and other blood vessels. Cardiovascular disease denote numerous conditions affecting the heart, blood, and vasculature of the body and encompasses, among others, coronary artery disease, angina pectoris, myocardial infarction, congestive heart failure, hypertension, cardiomyopathy, aortic stenosis, aneurysmal dilatation, peripheral vascular disease, and cerebrovascular disease.

A major indication of cardiovascular disease is “arteriosclerosis,” which is generally characterized by a thickening of the arterial walls and loss of elasticity. “Atherosclerosis” is a form of arteriosclerosis and refers to the formation of patchy subintimal thickening (i.e., atheromas or atherosclerotic plaques) of the arteries, which can lead to reduction or obstruction of blood flow. The atherosclerotic plaque is generally characterized by accumulated intracellular and extracellular lipids, smooth muscle cells, connective tissue, and glycosaminoglycans. Although the exact etiology of the disease is unknown, major risk factors for developing the disease include, among others, hyperlipidemia, hypertension, diabetes mellitus, obesity, hyperhomocysteinemia, physical inactivity, toxic insult (e.g., cigarette smoking) and pathogenic infections (e.g., Chlamydia pneumoniae infection).

In some embodiments, the cardiovascular disease that can be treated with the combination is coronary artery disease, which generally arises from subintimal deposition of atheromas in the arteries serving the heart. This coronary atherosclerosis is often irregularly distributed in different blood vessels, and can abruptly interfere with blood flow to segments of the myocardium because of rupturing of an eccentric atheromatous plaque. Coronary heart disease, overlaps with coronary artery disease, and is used interchangeably herein.

In some embodiments, the cardiovascular disease treatable with the combination is cerebrovascular disease. As used herein, “cerebrovascular disease” includes disorders in which an area of the brain is transiently or permanently affected by reduced arterial flow or ischemia, or bleeding and one or more of the cerebral blood vessels are involved in the disease condition. Transient ischemia is a transitory reduction in blood flow through a cerebral artery that can result in a momentary disturbance of brain function. The neurological deficit can include compromised cognitive function (e.g., slurred speech, difficulty reading, etc.), aphasia, numbness in limbs, headaches, or weakness/paralysis of a limb. In most instances, the neurological deficit is not permanent. In some embodiments, the cerebrovascular disease manifests as a stroke that progresses to permanent damage of brain and consequently impairment of brain function. Typical arteries involved include, among others, the anterior, middle and posterior cerebral arteries. As with other forms of cardiovascular disease, a major cause of cerebrovascular disease is atherosclerosis, or related diseases that lead to arterial obstruction. A less common cause is vascular inflammation. Related to cerebrovascular disease is carotid artery disease, which is atherosclerosis of the carotid arteries.

In some embodiments, the cardiovascular disease treated is peripheral vascular disease. As used herein, “peripheral vascular disease” refers to diseases resulting from the obstruction of large peripheral arteries and/or veins. Causes of peripheral vascular disease include, among others, atherosclerosis, inflammatory processes leading to stenosis, an embolism or thrombus formation. A common symptom is intermittent claudication, which is pain in the legs resulting from reduced blood flow. Another common symptom is postprandial abdominal angina caused by occlusion of one or more of the mesenteric arteries. Some cases of hypertension can arise from atherosclerotic occlusion of mesenteric arteries. Abdominal aortic aneurysms also can arise as a sequela of the atherosclerotic process. Presence of peripheral vascular disease is in many instances an indicator of more systemic cardiovascular disease, including coronary artery disease.

In some embodiments, the compositions and methods herein are used to treat or reduce the risk of a cardiovascular disease that is associated with a different, primary disease condition, such as autoimmune and/or inflammatory disease. In these embodiments, doses of the combination therapy can be administered to treat the associated, secondary condition or disease regardless of whether the underlying primary disease is treated. For example, as noted above, the autoimmune disease SLE is associated with increased atherosclerosis and cardiovascular disease such that they represent major causes of death in SLE patients. The risk of myocardial infarction increases by as much as 9 fold in patients with SLE. Hence, the combination therapy disclosed herein can be administered to treat or reduce the risk of atherosclerosis and cardiovascular disease associated with SLE, regardless of whether the doses are effective in treating the SLE. Other autoimmune diseases manifesting an increased vascular disease occurrence include, among others, diabetes mellitus (i.e., type I diabetes) and rheumatoid arthritis. Thus, in some embodiments, the combination therapy can be used in a method to treat or reduce the risk of cardiovascular disease, atherosclerosis, or other vascular disease in a subject with a pre-existing autoimmune disease, such as, for example, SLE, diabetes mellitus, or rheumatoid arthritis. In some embodiments, the combination therapy can be used to treat a subject when immunosuppressives other than IMPDH inhibitors, such as for example, cyclosporine, FK506, cyclophosphamide, and steroids, are administered as the primary medication to treat the autoimmune disease. The IMPDH inhibitor, in combination with the HMG CoA reductase inhibitor, can be administered at doses lower than those typically used to treat autoimmune disease, as a secondary measure to treat or delay the occurrence of cardiovascular disease, atherosclerosis, or other vascular diseases in the afflicted subject.

In some embodiments, the combination therapy is used to treated cardiovascular disease in subject with a transplanted organ. A frequent consequence of organ transplantation is arteriosclerosis of the transplanted organ. For example, cardiac allograft vasculopathy (CAV) is an accelerated form of coronary artery disease that occurs in heart transplant recipients. It is one of the leading causes of mortality among long-term cardiac transplant recipients. Long-term graft dysfunction arising from vasculopathies is similarly described in kidney, liver and lung transplant recipients. CAV is characterized by intimal proliferation and diffuse narrowing along the entire length of the blood vessels. Although mostly immune mediated, CAV is affected by nonimmune risk factors that include, among others, hyperlipidemia. In the methods herein, the combination therapy can be used to treat the organ transplant subject when immunosuppressives other than IMPDH inhibitors, such as for example, cyclosporine, FK506, cyclophosphamide, are prescribed to prevent the organ rejection. The IMPDH inhibitor, in combination with the HMG CoA reductase inhibitor, can be administered at doses lower than those typically used to prevent organ rejection, as a secondary measure to treat or delay the occurrence of transplant associated arteriosclerosis, and thereby prolong graft function.

In some embodiments, the combination therapy is used to treat vascular disease in subjects who are not organ transplant recipients. This population can include subjects afflicted with the conditions and diseases described above. In some embodiments, the subjects treated are healthy but have an increased risk or susceptibility to the diseases or associated conditions. For example, the subjects may have a genetic predisposition to the disease, as indicated by family history or genetic testing. In other embodiments, the subject may display one or more risk factors associated with an increased risk or susceptibility to the disease. As noted above, exemplary embodiments of markers or indications for increased risk of cardiovascular disease in humans include, among others, obesity, low HDL level, elevated cholesterol level, high fasting glucose, elevated blood pressure, and elevated levels of C-reactive protein, serum amyloid A, homocysteine, and inflammatory cytokines (e.g., interleukin-6, tumor necrosis factor-alpha, interleukin-8, etc.). Exemplary embodiments of markers for increased risk of autoimmune disease include, among others, presence of immuno reactive autoantibodies and corresponding autoantigens (see, e.g., Lernmark, A., 2001, J Clin Invest. 108:1091-1096), and an MHC type associated with autoimmune disease (see, e.g., Weyand and Goronzy, 2000, Arthritis Res. 2(3):203-4). As will be apparent to the skilled artisan, various other vascular diseases associated with arteriosclerosis and inflammatory vascular conditions can be treated with the combination of compounds herein.

Given the effects of the combination treatment on serum levels of cholesterol, triglycerides, and phospholipids, the present disclosure further provides methods of reducing the levels of these components in a subject. Accordingly, in some embodiments, the methods can be directed to reducing serum cholesterol levels in a subject in need thereof by administering an amount of an IMPDH inhibitor and a HMG CoA reductase inhibitor effective to lower serum cholesterol levels below those achievable with the compounds individually. As discussed above, the studies presented herein indicate that administration of IMPDH inhibitor MMF to Ldlr−/− mice had no effect on the total cholesterol or HDL cholesterol levels. However, when MMF is administered in combination with the statin simvastatin, there is a reduction in serum cholesterol levels beyond those observed when animals are treated with the statin alone. Thus, the combination of compounds can be administered to lower the serum cholesterol levels below those achievable by administration of the HMG CoA reductase inhibitor alone. The methods can be used to lower serum cholesterol levels by about 5% or more, 10% or more, 20% or more, 30% or more, 40% or more, 50% or more, 60% or more, 70% or more as compared to the levels in subjects prior to treatment, to a level considered to be desirable by those skilled in the art, as further described below.

In some embodiments, the combination is used to treat a subject with an abnormally elevated serum cholesterol level. The abnormally elevated cholesterol levels may be associated with a preexisting disease or be present in an otherwise healthy subject. As used herein, the term “abnormally elevated cholesterol level” refers to higher than levels considered to be acceptable by those skilled in the art for limiting the risk of atherosclerosis and other diseases associated with elevated cholesterol. Cholesterol levels can be measured in relation to total cholesterol or LDL cholesterol. Exemplary total cholesterol levels considered abnormally elevated in a human subject is 200 mg/dL and above, 240 mg/dL and above, or 280 mg/dL and above. Exemplary LDL cholesterol levels considered abnormally elevated in a human subject is 130 mg/dL and above, 140 mg/dL and above, 150 mg/dL and above, or 160 mg/dL and above. However, it is to be understood that what is determined to be an abnormal level of cholesterol can vary depending on risk factors, such as age, sex, family history and health condition, and can be assessed by those skilled in the art.

In some embodiments, the combination can be used in a method to lower serum triglyceride levels. In the studies described herein, the administration of a combination of MMF and simvastatin lowered serum triglyceride levels below those achievable with administration of each compound alone. The methods can be used to lower serum triglyceride levels by about 5% or more, 10% or more, 20% or more, 30% or more, 40% or more, 50% or more, 60% or more, or 70% or more, as compared to the level observed in the subjects prior to treatment, to a level considered to be desirable by those skilled in the art.

In some embodiments, the combination is used to treat a subject with an abnormally elevated serum triglyceride level. As used herein, “abnormally elevated triglyceride level” or “hypertriglyceridemia” refers to levels higher than those considered to be acceptable by those skilled in the art for limiting the risk of atherosclerosis and other diseases associated with elevated triglyceride levels. Triglyceride levels are generally measured as total serum triglyceride or VLDL triglyceride levels. Exemplary total triglyceride levels considered abnormally elevated in a human subject is 150 mg/dL and above, 160 mg/dL and above, 170 mg/dL and above, 180 mg/dL and above, or 200 mg/dL and above. As with cholesterol, an abnormal level of triglycerides can vary depending on risk factors, such as age, sex, family history and health condition, and can be assessed by those skilled in the art.

In some embodiments, the combination can be used in a method to lower serum phospholipid levels. In the studies described herein, the administration of a combination of MMF and simvastatin lowered serum phospholipids levels below those achievable with administration of each compound alone, and mirrored the effect of the combination on triglyceride levels. Thus, the methods can be used to lower serum phospholipid levels by about 5% or more, 10% or more, 20% or more, 30% or more, 40% or more, 50% or more, 60% or more, or 70% or more as compared to the level observed in the subjects prior to treatment, to a level considered to be desirable by those skilled in the art.

In view of the foregoing, the combination can be used to treat a subject afflicted with a disease or condition characterized by abnormally elevated cholesterol, triglyceride, and/or phospholipid levels. In some embodiments, the disease or condition is selected from, among others, obesity, metabolic syndrome (diabesity), insulin resistance, Type 2 diabetes, non-alcoholic fatty liver disease (NAFLD), alcoholic hepatosteatosis, hepatic cirrhosis, gout, hypothyroidism, nephritic syndrome, uremia, hyperuricemia, acute pancreatitis, chronic pancreatitis, obstructive liver disease, malignancy associated dysproteinemia, drug induced hypertriglyceridemia, dialysis associated hypertriglyceridemia, and familial hyperlipidemia.

In other embodiments, the combination is used to treat a subject afflicted with a disease or condition selected from, among others, transplant rejection associated hypertriglyceridemia, toxic chemical associated hepatic steatosis, Tangier disease, familial hypoalphalipoproteinemia, glucose-6-phosphate deficiency, glycogen storage disease, familial hypertriglyceridemia, sporadic hypertriglyceridemia, familial hypercholesterolemia, sporadic hypertriglyceridemia, primary hyperinsulinism, leprechaunism, hereditary pancreatitis, lipoprotein lipase deficiency, lipase-1-deficiency, RP1-associated hypertriglyceridemia, lecithin-cholesterol acyltransferase deficiency, familial combined hyperlipidemia, familial partial lipodystrophy, HIV-associated lipodystrophy, acquired partial lipodystrophy, autoimmunity associated lipodystrophy, familial hemophagocytic lymphohistiocytosis, congenital generalized lipodystrophy, insulin resistant diabetes mellitus, acanthosis nigricans, congenital leptin deficiency, Prader Willi Syndrome, Rabson-Mendenhall Syndrome, Alstrom Syndrome, Cohen Syndrome, POMC deficiency, monogenic obesity syndromes, idiopathic hepatosteatosis, fatty acid oxidation disorder, apolipoprotein C-II deficiency, apolipoprotein C-III deficiency, apolipoprotein E deficiency, apolipoprotein A-V deficiency, abetalipoproteinemia, hyperapobetalipoproteinemia, ataxia-telangiectasia, multiple symmetric lipomatosis, Mediastino-abdominal lipomatosis, erythropoietic-protoporphyria, Alagille syndrome, Sea Blue histiocyte disease, Niemann-Pick disease, cystic fibrosis, Wilson disease, alpha-1-antitrypsin deficiency, CD36 platelet glycoprotein VI deficiency, insulin receptor substrate-1 deficiency, LDHCP (lipoatrophy with diabetes, hepatic steatosis, hypertrophic cardiomyopathy and leukomelanodermic papules), carnitine palmytoyltransferase deficiency, and familial hyperchylomicronemia (resulting from a circulating inhibitor of lipoprotein lipase).

As will be apparent to the skilled artisan, the administration of MMF alone at doses lower than the doses used to lower the risk of allogenic organ transplant rejection resulted in the reduction of atheromatous plaques, along with decreases in serum triglyceride and phospholipid levels. Thus, the present disclosure further provides methods of treating certain vascular diseases as well as conditions of abnormally elevated serum triglyceride and phospholipids levels using MMF or MPA alone (i.e., monotherapy) at the doses disclosed herein.

In various embodiments, the therapies herein are directed to adult subjects. As used herein, “adult” in the context of human subjects refers to a person of about 18 years or older. As further described below, in some embodiments, the dosages administered are less than the dosages required to suppress the immune system for reducing the risk of organ rejection in an adult transplant patient. In some embodiments, the adult subjects may be further grouped into various age groups for purposes of treatment. For example, it is understood that as a human ages, there is an increased incidence of certain diseases that are “age related,” such as atherosclerosis, cardiovascular disease, arthritis, rheumatoid arthritis, and type II diabetes. Thus, older age groups can benefit from therapy with the combination therapy as compared to subjects in younger age groups. Grouping of adult subjects may also be useful for taking into consideration differences in metabolism of the statin and IMPDH inhibitory compounds by different age groups. Thus, in some embodiments, treatments with combination therapy can be directed to those in the group of about 65 years or older, in the group of about 50 to about 64 years of age, in the group of about 40 to about 49 years of age, and in the group of about 18 years to about 39 years of age. In some embodiments, the low dose of IMPDH inhibitor in combination with a HMG CoA reductase inhibitor can be used to delay the onset of such disease or lessen its severity in older patient populations that are at increased risk for such age related diseases, for example, patients who are 50 years or older.

In other embodiments, the treatments are directed to children and adolescents of about 18 years or younger, of about 12 years or younger, of about 6 years or younger, or of about 4 years or younger. Thus, in some embodiments, the low dose IMPDH inhibitor in combination with a HMG CoA reductase inhibitor can be administered to children and adolescents diagnosed with or at increased risk for vascular, autoimmune, and/or inflammatory diseases, and conditions associated therewith. For example, nearly a quarter of all systemic lupus cases are diagnosed in children, which may warrant early pharmacological intervention to limit the risk of developing atherosclerosis, cardiovascular disease, and other vascular diseases associated with SLE.

The compounds can be administered therapeutically to subjects who are suffering from the particular indication to achieve a therapeutic benefit. As used herein, “therapeutic benefit” includes, in addition to treating the underlying indication, reducing and/or ameliorating the overall number and/or severity of its associated symptoms and/or halting or slowing the progression of the indication and/or its symptoms. For example, as described above, the combination of IMPDH and HMG CoA reductase inhibitors can be administered therapeutically to individuals afflicted with an indication to avoid the onset of symptoms or side-effects associated with the indication, regardless of whether the underlying the indication is treated. In other embodiments, the combination therapy can be administered prophylactically to subjects that are not suffering from the particular indication, including healthy subjects, to achieve prophylactic benefit.

Generally, the compounds are administered in amounts that, in combination, provide therapeutic and/or prophylactic benefit. The actual dosage of each class of compound will vary, depending upon, among other factors, the individual, the condition being treated, the state of the disease, and other factors that will be apparent to the prescribing physician, such as age, sex, and weight. Those skilled in the art will be able to select a proper amount of the compounds based on Table 1, below, on the rest of this disclosure, and on the disease to be treated. An important practical effect of such combinations is to facilitate patient compliance. Thus, the dose of HMG CoA reductase inhibitor may use a dose sufficient to provide the desired anti-inflammatory, immunomodulatory, and therapeutic effect, which in some embodiments are the dosages normally used to treat hypercholesterolemia. For instance, simvastatin in oral dosage form is prescribed at 5-40 mg/day while fluvastatin is prescribed at 20-80 mg/day for an adult. Thus, in some embodiments, the dose of statin administered can be about 5 mg/day to about 100 mg/day or 20 mg/day to about 80 mg/day. Exemplary doses of statins to treat a human subject include, among others, 5, 10, 15, 20, 25, 30, 40, 50, 60, 70, 80, 90 and 100 mg/day. The equivalent doses in other mammals are further described below.

In some embodiments, the statin is administered at a dose if administered alone is effective to lower serum cholesterol level by about 5%, about 10%, by about 20%, about 40%, about 50%, about 60% as compared to the level observed in the subjects prior to treatment, to a level considered to be desirable by those skilled in the art. An exemplary level desirable in a human subject is below 200 mg/dL, such as 140 to 180 mg/dL. It is well within the skill of those in the art to determine the dose of statin required for a specified decrease in cholesterol levels.

It is to be understood that in some embodiments, lower dosages may be used since the effect of statins on inflammation and the immune system may occur in the absence of lowering, significant lowering, or substantial lowering of serum cholesterol level. As used herein, a “lowering” of serum cholesterol level refers to a decrease of about 5% or more serum cholesterol level as compared to levels in untreated subjects. A “significant lowering” refers to a decrease of about 25% or more serum cholesterol level as compared to levels in untreated subjects. A “substantial lowering” refers to a decrease of about 40% or more of serum cholesterol levels as compared to levels in untreated subjects. Exemplary dosages for human subjects that are below the normal doses typically used to lower cholesterol levels are less than 10 mg/day, including, among others, about 1, 2, 3, 5, 8, and about 9 mg/day, depending on the specific statin and the individual being treated.

Similarly, the dose of the IMPDH inhibitor used is an amount sufficient to effect treatment of the specified disorder in combination with the HMG CoA reductase inhibitor. In some embodiments, the dose of the IMPDH inhibitor is an amount sufficient to reduce the risk of allograft rejection. The recommended dose of CellCept® is 1 g administered orally or intravenously twice daily for renal transplant (i.e., a daily dose of 2 g; corresponding to a daily dose in the range of about 20-45 mg/kg for a patient body mass in the range of 45-100 kg) and 1.5 g administered orally or intravenously twice daily for hepatic and cardiac transplant (i.e., a daily dose of 3 g; corresponding to a daily dose of about 30-67 mg/kg for a patient body mass in the range of 45-100 kg). The recommended dose of Myfortic® is 720 mg administered orally twice daily (i.e., a daily dose of 1.44 g; corresponding to a daily dose in the range of about 14-32 mg/kg for a patient body mass in the range of 45-100 kg). Accordingly, in these embodiments, the dose of IMPDH inhibitor administered can be a dose equivalent to a human dose of about 2 g/day to about 3 g/day of MMF, or a corresponding dose of MPA, which can be readily determined by the skilled artisan.

In some embodiments, the dose of the IMPDH inhibitor used may be lower than the standard dosage typically administered to reduce the risk of allograft rejection. At these lower dosages, therapeutic and/or prophylactic benefit can be achieved while avoiding or minimizing the adverse consequences of severe immunosuppression that occurs with standard doses of such compounds. These levels of IMPDH inhibitor can be achieved with doses taken once or more per day.

In some embodiments, the dose of IMPDH inhibitor administered can be a dose equivalent to a human dose of above 1 g/day to less than about 2 g/day of MMF (e.g., 1.10, 1.20, 1.30, 1.40, 1.50, 1.75, and 1.90 g/day), or a corresponding dose of MPA. These doses correspond to about 11 to about 42 mg/kg/day, depending on patient body mass, including 11 to 24, 11 to 27, 11 to 29, 11 to 31, 11 to 33, and 11 to 39 mg/kg/day. In some embodiments, the dose is about 11, 12, 14, 16, 18, 20, 22, 25, 30, 35, 39, and 42 mg/kg/day.

In some embodiments, the dose of IMPDH inhibitor administered can be a dose equivalent to a human dose of about 1.0 g/day or less. In some embodiments, the dose of MMF administered can be a dose equivalent to a human dose of about 0.5 to about 1.00 g/day of MMF (e.g., 0.5, 0.6, 0.75, and 1.0 gm/day), or a corresponding dose of MPA. These doses correspond to about 5 to about 22 mg/kg/day, depending on patient body mass, including 5 to 11, 5 to 13, and 5 to 17 mg/kg/day. In some embodiments, the dose is about 5, 6, 8, 10, 12, 14, 16, 18, 20, or 22 mg/kg/day.

In some embodiments, the dose of IMPDH inhibitor administered can be a dose equivalent to a human dose about 100 to less than about 500 mg/day of MMF (e.g., 100, 125, 150, 175, 200, 225, 250, 275, 300, 325, 350, 375, 400, 425, 450, and 475 mg/day), or a corresponding dose of MPA. These doses correspond to a human dose of about 1.1 to about 11 mg/kg/day, depending on patient body mass, including 1.0 to 2.2, 1.1 to 2.8, 1.1 to 3.3, 1.1 to 3.9, 1.1 to 4.4, 1.1 to 5.0, 1.1 to 5.6, 1.1 to 6.1, 1.1 to 6.7, 1.1 to 7.2, 1.1 to 7.8, 1.1 to 8.3, 1.1 to 8.9, 1.1 to 9.4, 1.1 to 10.0, and 1.1 to 10.6 mg/kg/day. In some embodiments the dose is about 1.5, 2, 3, 4, 5, 7, 9 or 11 mg/kg/day.

In some embodiments, the dose of IMPDH inhibitor administered can be a dose equivalent to a human dose about 250 to less than about 500 mg/day of MMF (e.g., 250, 275, 300, 325, 350, 375, 400, 425, 450, and 475 mg/day), or a corresponding dose of MPA. These doses correspond to a human dose of about 2.5 to about 11 mg/kg/day, depending on patient body mass, including 2.5 to 5.6, 2.5 to 6.1, 2.5 to 6.7, 2.5 to 7.2, 2.5 to 7.8, 2.5 to 8.3, 2.5 to 8.9, 2.5 to 9.4, 2.5 to 10.0, and 2.5 to 10.6 mg/kg/day. In some embodiments, the dose is about 3, 4, 5, 7, 9, or 11 mg/kg/day.

In some embodiments, the dose of IMPDH inhibitor administered can be a dose equivalent to a human dose about 100 to less than about 250 mg/day (e.g., 100, 125, 150, 175, 200, and 225 mg/day), or a corresponding dose of MPA. These doses correspond to a human dose of about 1.0 to about 5.0 mg/kg/day, depending on patient body mass, including 1.0 to 2.2, 1.0 to 2.8, 1.0 to 3.3, 1.0 to 3.9, and 1.0 to 4.4 mg/kg/day. In some embodiments, the dose is about 1, 2, 3, 4, or 5 mg/kg/day.

In some embodiments, the dose of IMPDH inhibitor administered can be a dose equivalent to a human dose of about 5 to less than about 100 mg/day of MMF (e.g., 5, 10, 20, 30, 40, 50, 60, 70, 80, and 90 mg/day), or a corresponding dose of MPA. These doses correspond to a human dose of about 0.05 to about 2.0 mg/kg/day, depending on patient body mass, including 0.05 to 0.11, 0.05 to 0.22, 0.05 to 0.44, 0.05 to 0.67, 0.05 to 0.89, 0.05 to 1.11, 0.05 to 1.33, 0.05 to 1.56, 0.05 to 1.80, 0.05 to 2.00 mg/kg/day. In some embodiments, the dose is about 0.05, 0.1, 0.2, 0.3, 0.4, 0.5, 0.6, 0.7, 0.8, 0.9, 1.0, 1.2, 1.4, 1.6, 1.8, or 2.0 mg/kg/day.

In some embodiments, the dose of IMPDH inhibitor administered can be a dose equivalent to a human dose above about 50 to less than about 100 mg/day (e.g., 55, 60, 70, 80, and 90 mg/day) of MMF, or a corresponding dose of MPA. These doses correspond to a human dose of about 0.55 to about 2.0 mg/kg/day, depending on patient body mass, including 0.55 to 1.22, 0.55 to 1.33, 0.55 to 1.56, 0.55 to 1.80, and 0.55 to 2.00 mg/kg/day. In some embodiments, the dose is about 0.55, 0.6, 0.7, 0.8, 0.9, 1.0, 1.2, 1.4, 1.6, 1.8, or 2.0 mg/kg/day.

In some embodiments, the dose of IMPDH inhibitor administered can be a dose equivalent to a human dose of about 5 to about 50 mg/day of MMF (e.g., 5, 10, 20, 25, 30, 40, and 50 mg/day), or a corresponding dose of MPA. These doses correspond to a human dose of about 0.05 to about 1.1 mg/kg/day, depending on patient body mass, including 0.05 to 0.11, 0.05 to 0.22, 0.05 to 0.33, 0.05 to 0.44, 0.05 to 0.56, 0.05 to 0.67, 0.05 to 0.89, and 0.05 to 1.11 mg/kg/day. In some embodiments, the dose is about 0.05, 0.1, 0.2, 0.3, 0.4, 0.5, 0.6, 0.7, 0.8, 0.9, 1.0, or 1.1 mg/kg/day.

It is to be understood that while the dosages used in the animal model studies below are given for administration to mice, equivalent doses for human subjects (and other mammals) can be determined by those skilled in the art. Likewise, human doses can be used to determine the equivalent doses for other animals (e.g., mice). Determining the equivalent doses is well within the skill of those in the art. For instance, the therapeutically effective dose in an animal can be determined from in vitro as well as in vivo analysis. For example, the ED₅₀ for immunosuppressive effect of MMF or MPA can be examined in vitro using inhibition of antibody response to sheep red blood cells (see, e.g., Eugui et al., 1991, “Lymphocyte-selective antiproliferative and immunosuppressive effects of mycophenolic acid in mice,” Scandinavian J. Immunol. 33, 175-183) while in vivo experiments can be based on allograft survival (see, e.g., Hao et a., 1990, “RS-61443 (MMF) allows islet allografting and specific tolerance induction in adult mice,” Transplantation Proceedings 22(2):876). Such studies indicate that in mice, the ED₅₀ for inhibiting the antibody reaction is about 33 mg/kg/day while the effective dose for in vivo prolongation of allograft survival is about 80 mg/kg/day. Given these animal doses, the equivalent human dose can be estimated based on body surface area (see, e.g., Ng, R., 2004, “Drug Development and Preclinical

Studies.” In Drugs: From Discovery to Approval, Chapter 5, Wiley-Liss). For example, converting from dose in mg/kg to a dose in mg/m² for mice is to multiply the mg/kg dose by 3 kg/m². The equivalent human dose can be calculated by using the conversion factor of 37 kg/m². For example, the correspondence between MMF doses administered to a mouse and an equivalent human dose (for a reference weight of 60 kg for a human) is given below.

| | |
|---|---|
| Mouse | Human |

| | | | |
|---|---|---|---|
| 6 | mg/kg/day | 30 | mg/day |
| 15 | mg/kg/day | 75 | mg/day |
| 30 | mg/kg/day | 150 | mg/day |
| 100 | mg/kg/day | 500 | mg/day |

In some embodiments, the daily doses described above can be achieved by administering the MMF or MPA in unit dosage amounts, or, alternatively, the daily doses may be achieved by administering MMF or MPA in two or more equal or unequal dosage amounts during the course of the day, such that the total amount of MMF or MPA administered per day equals the total amount desired.

Similarly, the equivalent doses of a statin can be determined in a similar manner as described for MMF and MPA. For example, dose of simvastatin used for atheroprevention in the mouse studies described below is 90 mg/kg/day, which is 270 mg/m² (90 mg/kg×3 kg/m²). Taking into consideration the human conversion factor of 37 kg/m² and a reference human weight of 60 kg, the human equivalent dose is 438 mg/day. Although this dose is higher than the standard human doses, mice on a high-cholesterol diet have been shown to be less susceptible to the effects of statins than humans because mice are able to upregulate HMG CoA reductase, the enzyme targeted by simvastatin, by up to 8-fold when treated with an HMG CoA reductase inhibitor (see, e.g., Kita et al., 1980, J Clin. Invest. 66:1094-1100). Thus, in order for bioequivalence to be achieved, higher doses of statins are required in mice than would be used in humans. For example, Johnston et al., 2001, Intl J. Pharmac. 229:75-86, used dosages of 70 mg/kg/day in mice, equivalent to 340 mg/day in a human, which is over 4 times the standard statin dose. Similarly, Bisgaier et al., 1997, J Lipid Res. 38:2502-2515, used simvastatin at 100 mg/kg/day in mice, and demonstrated a decrease in total plasma cholesterol and LDL cholesterol with no toxicity at this dose.

According to the methods described herein, the IMPDH and HMG CoA reductase inhibitors are adjunctively administered to the subject. Relative to the administration of the IMPDH inhibitor, the adjunctive administration of the HMG-CoA reductase inhibitor can occur before, at the same time (e.g., contemporaneously), subsequent to, or on an irregular basis. Those skilled in the art are able to identify a suitable temporal relationship between the agents which will achieve the desired treatment result, several examples of which are set out below. Treatment may continue until the disease process is resolved, until the symptoms of the disease are reduced to a satisfactory level, or until otherwise determined by the physician and patient. In some cases, treatment may be chronic.

When administered non-contemporaneously (e.g., sequentially) the active agents can typically be formulated separately, using off-the shelf formulations. When administered contemporaneously, it may be advantageous to provide the HMG-CoA reductase inhibitor in a dosage form in combination with the IMPDH inhibitor, as disclosed in the compositions described below, to improve patient compliance and the effect of the therapy, although off-the-shelf formulations may be used.

An embodiment of the disclosure is a convenient dosage form of an IMPDH inhibitor and an HMG-CoA reductase inhibitor. The composition may take the form of a pill, capsule or tablet that comprises an effective amount of both active ingredients. Effective amounts of a variety of combinations are set out in Table 1, below. Based on this disclosure, those skilled in this art are able to identify further effective dosage combinations, all of which are included in the instant disclosure.

In some embodiments, the combination composition includes amounts of IMPDH and HMG CoA reductase inhibitors that correspond to unit or fractional amounts of their standard daily dosages. Standard daily dosages recommended for specific IMPDH and HMG CoA reductase inhibitors can be obtained from The 2005 Edition of The Physician's Desk Reference (“PDR”), incorporated herein by reference. In some embodiments, the combination composition includes an amount of an IMPDH inhibitor that corresponds to a unit or fractional unit amount of the specified inhibitor above (d.

The pill, capsule or tablet may optionally contain, along with an effective amount of both active ingredients, a diluent such as lactose, sucrose, dicalcium phosphate, and the like; a disintegrant such as starch or derivatives thereof like pregelatinized starch (corn); a lubricant such as magnesium stearate and the like; and a binder such as starch, gum acacia, polyvinylpyrrolidone, gelatin, cellulose and derivates thereof, and the like. Additional inactive ingredients may include butylated hyrdoxyanisole NF, citric acid monohydrate USP, croscarmellose sodium NF, hydroxypropyl cellulose; hydroxypropyl methylcellulose USP, iron oxides, lactose monohydrate NF, magnesium stearate NF, potassium bicarbonate, povidone, povidone K-90, ammonium hydroxide, microcrystalline cellulose NF, Opadry White YS-1-7040, polyethylene glycol, PEG 8000, sodium lauryl sulfate, polysorbate 80 NF, simethicone emulsion, talc, titanium dioxide, calcium carbonate USP, candelilla wax FCC; FD&C Blue 2, D&C Yellow 10, ethyl alcohol, methyl alcohol, n-butyl alcohol, propylene glycol, shellac and propyl gallate NF.

Non-limiting examples of excipiating agents include benzyl alcohol, black iron oxide, butylparaben, edentate calcium disodium, methylparaben, propylparaben, and sodium propionate.

Compositions that are in the form of tablets may include optional coatings designed, for example, to be resistant to the acid environment of the stomach and remain undissolved until they reach the alkaline environment of the small intestine. Films that dissolve between pH 5.5 and 6.5 are generally preferred. A wide variety of such coatings are known to those skilled in the art.

Liquid pharmaceutically administrable compositions can be prepared by dissolving, dispersing, etc. the active compounds (each about 0.5% to about 40%), as described above, and optional pharmaceutical adjuvants in a carrier, such as for example, water, saline, aqueous dextrose, glycerol, ethanol and the like, to form a solution or suspension. In 5% dextrose solution, MMF has a solubility of 65.8 mg/ml and a pH of 2.4 to 4.1. Inactive ingredients in liquid formulation may further include aspartame, citric acid anhydrous, colloidal silicon dioxide, mixed fruit flavor, sodium citrate dehydrate, sorbitol, soybean lecithin and xanthan gum.

If desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, etc.

Methods of preparing the various dosage forms discussed are known, or will be apparent, to those skilled in this art; for example, see Remington's Pharmaceutical Sciences, 2000, 20th edition (Mack Publishing Company, Easton, Pa.).

Mycophenolate mofetil, or morpholinoethyl E-6-(1,3-dihydro-4-hydroxy-6-methoxy-7-methyl-3-oxo-5-isobenzofuranyl)-4-methyl-4-hexenoate, can be made as described in U.S. Pat. No. 4,753,935. The pharmaceutically acceptable salts or derivatives of mycophenolate mofetil can be made as described in U.S. Pat. Nos. 4,686,234; 4,725,622; 4,727,069; 4,748,173; 4,753,935; 4,786,637; 4,808,592; 4,861,776; 4,868,153; 4,948,793 4,952,579; 4,959,387; and 4,922,467. The methods for synthesis of HMG CoA reductase inhibitors are described in the references disclosed above. Formulating the combination of the active and inactive ingredients into the desired pill, tablet, capsule or liquid can be achieved according to known methods standard in the art.

Also provided are kits containing an IMPDH inhibitor packaged alone or together with an HMG-CoA reductase inhibitor. The kits may comprise one or more containers filled with solid compositions or aqueous solutions of an IMPDH inhibitor and an HMG-CoA reductase inhibitor, optionally in a combined container. For example, a kit may comprise one bottle containing a dosage form of an IMPDH inhibitor, and a separate bottle comprising a dosage form of an HMG-CoA reductase inhibitor. Alternatively, the kit may comprise a device for administering the active therapeutic agents in combination with each other, such as in a combination pill. In this case, the kit would comprise, for example, a single bottle or a blister pack comprising a dosage form comprising a combination of a an IMPDH inhibitor and HMG-CoA reductase inhibitor. Other devices for administration include, among others, a graduated cup or cylinder, a dropper, or a syringe. A kit may optionally include instructions for proper dosing use and information on the drug. Mediums for this information include printed forms, compact disc, flash memory, videotape, or other as is known in the art.

In some embodiments, the combination therapies may be directed to prevention of restenosis, which is a form of chronic vascular injury leading to vessel wall thickening and loss of blood flow to the tissue supplied by the blood vessel. It typically occurs in response to vascular reconstructive procedures, including virtually any manipulation which attempts to relieve vessel obstructions.

The combination therapy may be provided to prevent restenosis in a subject undergoing vascular reconstructive procedures, either by oral administration or by local administration to the site of the procedure, either by injection or other direct application, or in conjunction with a stent.

In some embodiments, the therapy is administered in conjunction with stents, which comprise a generally tubular structure (which includes for example, spiral or coil shapes), the surface of which is coated with the IMPDH and HMG-CoA reductase inhibitors. Typically, a stent is a scaffolding, usually cylindrical in shape, that may be inserted into a body passageway (e.g., bile ducts) or a portion of a body passageway, which has been narrowed, irregularly contoured, obstructed, or occluded by a disease process (e.g., ingrowth by an atherosclerotic plaque) in order to prevent closure or reclosure (restenosis) of the passageway. Stents act by physically holding open the walls of the body passage into which they are inserted.

A variety of stents may be used with the therapeutic combination disclosed herein, including for example, esophageal stents, vascular stents, biliary stents, pancreatic stents, ureteric and urethral stents, lacrimal stents, Eustachian tube stents, fallopian tube stents, and tracheaUbronchial stents. Stents may be readily obtained from commercial sources, or constructed in accordance with well-known techniques. Representative examples of stents are described in U.S. Pat. Nos. 4,768,523; 4,776,337; 5,041,126; 5,052,998; 5,064,435; 5,089,606; 5,147,370; 5,176,626; 5,213,580; and 5,328,471; the disclosures of which are incorporated herein by reference.

The stents may be coated with the IMPDH inhibitor and HMG-CoA reductase inhibitor in a variety of manners, including for example: (a) by directly affixing to the stent these therapeutic agents (e.g., by either spraying the stent with a polymer/drug film, or by dipping the stent into a polymer/drug solution), (b) by coating the stent with a substance such as a hydrogel which will in turn absorbs the therapeutic agents, (c) by interweaving a thread coated with the therapeutic agents (or the polymer itself formed into a thread) into the stent structure, (d) by inserting the stent into a sleeve or mesh which is comprised of or coated with the therapeutic agents, or (e) constructing the stent itself from a composition comprising the therapeutic agents. In some embodiments, the composition should firmly adhere to the stent during storage and at the time of insertion, and should not be dislodged from the stent when the diameter is expanded from its collapsed size to its full expansion size. The therapeutic agents should not degrade during storage, prior to insertion, or when warmed to body temperature after expansion inside the body. In addition, it should preferably coat the stent smoothly and evenly, with a uniform distribution of therapeutic agent, while not changing the stent contour. Embodiments of therapeutic agents in the stent should provide a uniform, predictable, prolonged release of the therapeutic agents into the tissue surrounding the stent once it has been deployed. For vascular stents, in addition to the above properties, the composition should not render the stem thrombogenic (causing blood clots to form), or cause significant turbulence in blood flow (more than the stent itself would be expected to cause if it was uncoated). Standard methods for coating stents are generally known and examples can be found in U.S. Pat. Nos. 6,153,252; 6,258,121; and 5,824,048, herein incorporated by reference.

For each of the therapeutic compounds listed in the present application, the amount of therapeutic agent used will be dependent upon the particular drugs employed. Typically, the amount of drug can represent about 0.001% to about 70%, about 0.00 1% to about 60%, or about 0.00 1% to about 45% by weight of the coating.

To achieve therapeutic and/or prophylactic benefit, an effective amount of the IMPDH inhibitor and HMG-CoA reductase inhibitor should be applied to the site of vascular reconstruction such that restenosis is prevented. In general, for treating humans or animals, between approximately 0.1 mg/kg to 500 mg/kg body weight, between approximately 1 mg/kg to 50 mg/kg body weight, or between approximately from 1 mg/kg to 25 mg/kg body weight of the therapeutic compound can be used. Depending upon the half-life of the compound in the particular animal or human, the compound can be administered on a variety of schedules, including once (e.g., in conjunction with a delayed release stent), several times per day, once a week or as otherwise desired. The methods disclosed herein provide for single as well as multiple administrations, given either simultaneously (e.g., in conjunction with a stent implant procedure) or over an extended period of time.

Within another aspect of the present disclosure, methods are provided for expanding the lumen of a body passageway, comprising inserting a stent into the passageway, the stent having a generally tubular structure, the surface of the structure being coated with an IMPDH inhibitor and HMG-CoA reductase inhibitor such that the passageway is expanded. In various embodiments, the lumen of a body passageway is expanded in order to eliminate a vascular obstruction.

Generally, stents are inserted in a similar fashion regardless of the site or the disease being treated. Briefly, a preinsertion examination, usually a diagnostic imaging procedure, endoscopy, or direct visualization at the time of surgery, is generally performed in order to determine the appropriate positioning for stent insertion. A guidewire is then advanced through the lesion or proposed site of insertion, and over this is passed a delivery catheter which allows a stent in its collapsed form to be inserted. Typically, stents are capable of being compressed, so that they can be inserted through tiny cavities via small catheters, and then expanded to a larger diameter once they are at the desired location. Once expanded, the stent physically forces the walls of the passageway apart and holds them open. As such, they are capable of insertion via a small opening, and yet are still able to hold open a large diameter cavity or passageway. The stent may be self-expanding (e.g., the Wallstent and Gianturco stents), balloon expandable (e.g., the Palmaz stent and Strecker stent), or implanted by a change in temperature (e.g., the Nitinol stent).

To enhance its drug carrying and drug eluting properties, the stent may be coated with a wide variety of polymeric carriers, including for example both biodegradable and non-biodegradable compositions. Representative examples of biodegradable compositions include albumin, gelatin, starch, cellulose, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters) (see generally, ilium, L. and Davids, S. S., 1987, “Polymers in controlled Drug Delivery” Wright, Bristol; Arshady, 1991, J. Controlled Release 17:1-22; Pitt, 1990, hit. J. Phar. 59:173-196; and Holland et al., 1986, J. Controlled Release 4:155-0180). Representative examples of nondegradable polymers include EVA copolymers, silicone rubber and poly (methylmethacrylate). Exemplary polymeric carriers include poly (ethylene-vinyl acetate)(40% cross-linked), poly(D,L-lactic acid) oligomers and polymers, poly(L-lactic acid) oligomers and polymers, poly(glycolic acid), copolymers of lactic acid and glycolic acid, poly(caprolactone), poly (valerolactone), polyanhydrides, copolymers of poly(caprolactone) or poly(lactic acid) with polyethylene glycol and blends thereof.

Polymeric carriers may be fashioned in a variety of forms, including for example, coils, expandable coils, rod-shaped devices, pellets, slabs, or capsules (see, e.g., Goodell et al., 1986, Am J Hosp Pharm. 43:1454-1461; Langer et al., 1980, “Controlled release of macromolecules from polymers”, in Biomedical polymers, Polymeric materials and pharmaceuticals for biomedical use (Goldberg, E. and Nakagim, A. eds.) Academic Press, pp. 113-137; Rhine et al., 1980, J. Pharm. Sci. 69:265-270; Brown et al., 1983, J. Pharm. Sci. 72:1181-1185; and Bawa et al., 1985, J. Controlled Release 1:259-267). The IMPDH and/or HMG CoA reductase inhibitors may be linked to the polymeric carrier by occlusion in the matrices of the polymer, bound by covalent linkages, or encapsulated in microcapsules. Within certain embodiments, compositions are provided in non-capsular formulations such as microspheres (ranging from nanometers to micrometers in size), pastes, threads of various sizes, coils, films and sprays.

The composition should be biocompatible, and release the inhibitors over a period of several days to months. For example, “quick release” or “burst” compositions are provided that release greater than 10%, 20%, or 25% (w/v) of the loaded inhibitors over a period of 7 to 10 days. Such “quick release” compositions should, within certain embodiments, be capable of releasing chemotherapeutic levels (where applicable) of the therapeutic agents. Within other embodiments, “low release” compositions are provided that release less than 1% (w/v) of the loaded therapeutic agents over a period of 7 to 10 days. Further, compositions should preferably be stable for several months and capable of being produced and maintained under sterile conditions.

### 6. EXAMPLES

### 6.1 Example 1

### Doses of Combination Therapies

Treatment regimens comprising combination therapy likely will be most effective by approximately daily dosing of the two active agents, although other dosing schedules are possible. The preferred manner of administration, for the conditions detailed herein, is oral using a convenient daily dosage regimen that can be adjusted according to the degree of the disease. Preferred combinations, which are not intended to limit the scope of options set out herein, are listed in Table 1.

TABLE 1
Therapeutically Effective Combination Doses

| Combination | Dosage frequency/Dose Amount |
|---|---|
| MMF | Standard dose, 0.5 to 1.0 g/day, |
| | 100 to <500 mg/day, |
| | 5 to <100 mg/day |
| atorvastatin (Lipitor ®) | 10, 20, 40, 80 mg/day |
| MMF | Standard dose, 0.5 to 1.0 g/day, |
| | 100 to <500 mg/day, |
| | 5 to <100 mg/day |
| fluvastatin (Lescol ®) | 20 to 40 mg/day |
| MMF | Standard dose, 0.5 to 1.0 g/day, |
| | 100 to <500 mg/day, |
| | 5 to <100 mg/day |
| lovastatin (Mevacor ®) | 10, 20, 40 mg/day |
| MMF | Standard dose, 0.5 to 1.0 g/day, |
| | 100 to <500 mg/day, |
| | 5 to <100 mg/day |
| pravastatin (Pravachol ®) | 10, 20, 40, 80 mg/day |
| MMF | Standard dose, 0.5 to 1.0 g/day, |
| | 100 to <500 mg/day, |
| | 5 to <100 mg/day |
| simvastatin (Zocor ®) | Single daily dose in evening, |
| | with or without food |
| | 10, 20, 40, 80 mg/day |
| MMF | Standard dose, 0.5 to 1.0 g/day, |
| | 100 to <500 mg/day, |
| | 5 to <100 mg/day |
| rosuvastatin (Crestor ®) | 10, 20, 40 mg/day |

In the above chart, the dose of MMF is selected to be in the range of from 0.005 to 5.0 g/day, such as, for example, 0.015, 0.03, 0.05, 0.075, 0.10, 0.15, 0.30, 0.50, 1.0, 1.5, 2.0, 2.5, 3.0, or 3.5 g/day, which may be alternatively divided into equal or unequal doses during the course of the day, such that the combination of doses per day equals the total amount desired per day.

In an embodiment of a combination composition, the composition is a tablet comprising MMF (0.015, 0.03, 0.05, 0.075, 0.10, 0.15, 0.30, 0.50, 1.0, 1.5, or 2.0 g) and simvastatin (Zocor®) (10, 20, 40 or 80 mg). In another embodiment, the tablet comprises MMF (0.015, 0.03, 0.05, 0.075, 0.10, 0.15, 0.30, 0.50, 1.0, 1.5, or 2.0 g) and simvastatin (Zocor®) (5, 10, 20, 40 or 80 mg). Alternatively, the embodiment comprises MMF (0.015, 0.03, 0.05, 0.075, 0.10, 0.15, 0.30, 0.50, 1.0, 1.5, or 2.0 g) and atorvastatin (Lipitor®) (10, 20, 40 or 80 mg). Each embodiment may be administered from one to four times per day. Each embodiment may have inactive ingredients or an enteric coating as provided herein. An important practical effect of the compositions and methods described herein is that it overcomes the challenge faced by clinicians who are hesitant to use statins in lupus patients as they may have the adverse effects of worsening fibromyalgia or cause complications through the adverse effect of fibromyositis. The combination therapy described herein provides relief from the concerns about these side-effects.

In some methods or compositions described herein, it may be desirable to employ a dose of a statin which falls below the regularly accepted dosages (listed in Table 1). The disclosure thus contemplates doses of the statins which are as low as 5% of the lowest approved dosage for the statin, including, among others, 75%, 50%, 25% or 10% of the lowest approved dosage.

### 6.2 Example 2

### Effect of Low Dose MMF on a Mouse Model of Atherosclerosis

This example illustrates the effect of low-dose MMF on low-density lipoprotein receptor deficient mice, B6.129S7-Ldlrtm1Her/J, hereinafter referred to as Ldlr−/− mice, obtained from Jackson Labs. When placed on a high-fat, high-cholesterol “Western-type” diet (WTD), these mice develop severe atherosclerosis (Ishibashi et al., 1994, J Clin Invest. 93(5):1885-93).

Study Design: Male mice were fed a high-fat, high-cholesterol “Western-type” diet (15% cocoa butter, 0.25% cholesterol by weight, 34% of calories from fat (“Diet W” Hope Farms, Woerden, Nertherlands) for 12 weeks, a time which has been shown to be sufficient for the mice to develop significant atherosclerosis, as assessed by the appearance of atheromatous lesions at the aortic root.

Male Ldlr−/− mice consume, on average, 3.0 to 3.3 grams of Diet W per mouse per day. Diet W was formulated with CellCept® incorporated directly into the chow, at concentrations of 0.0064%, 0.0134%, 0.025% or 0.08% mycophenolate mofetil by weight. When administered in this way, the mice consume 6 mg/kg/day, 15 mg/kg/day, 30 mg/kg/day, or 100 mg/kg/day of the drug, respectively. Animals were sacrificed 12 weeks after feeding on the study diet, and the endpoints determined.

The primary endpoint was evaluated using histological analysis of plaque area at the aortic root. The total lesion area in oil red O-stained cryostat sections of the aortic root was quantified using a Leica image analysis system. Mean lesion area (as a percentage of aortic cross-sectional area) was calculated from 4 oil red O-stained sections, beginning at the appearance of the tricuspid valves.

The average atherosclerotic area was compared between the groups using the one-way ANOVA test. Additional secondary endpoints examined were plasma levels of cholesterol, HDL cholesterol, triglycerides, and phospholipids.

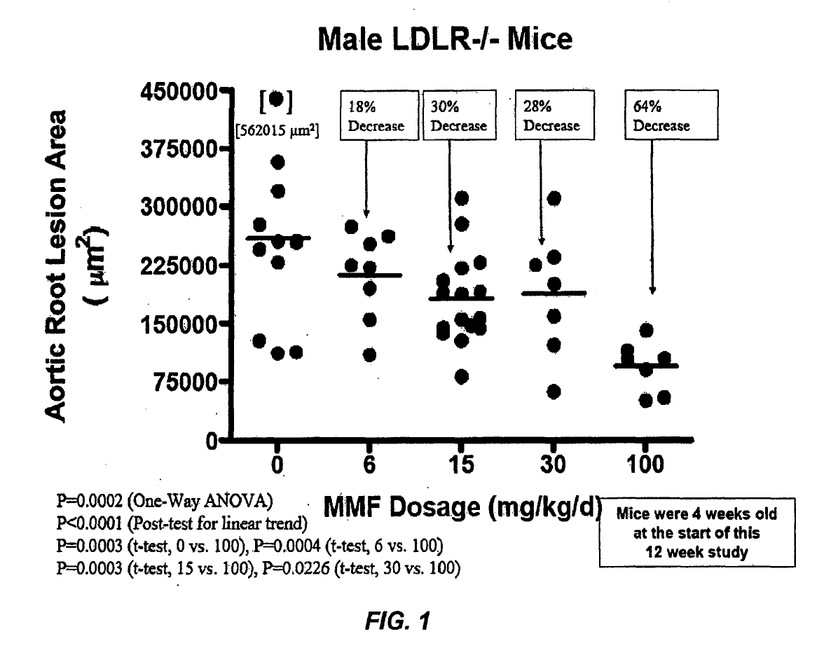

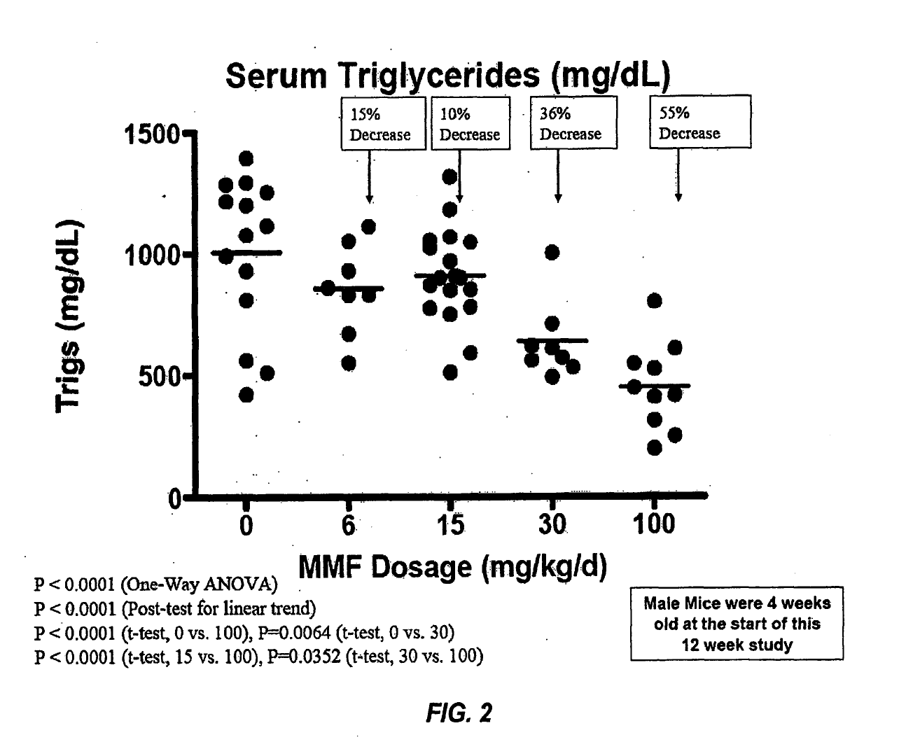

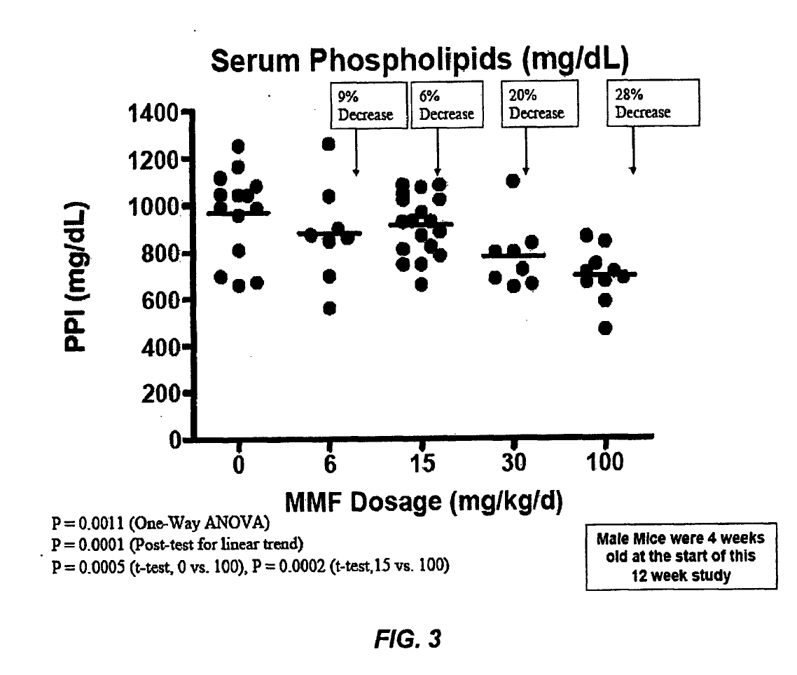

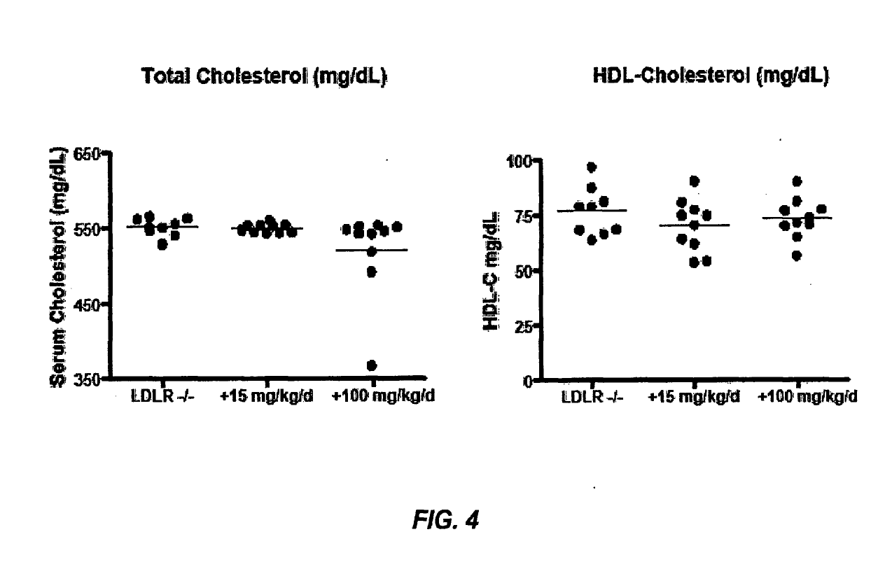

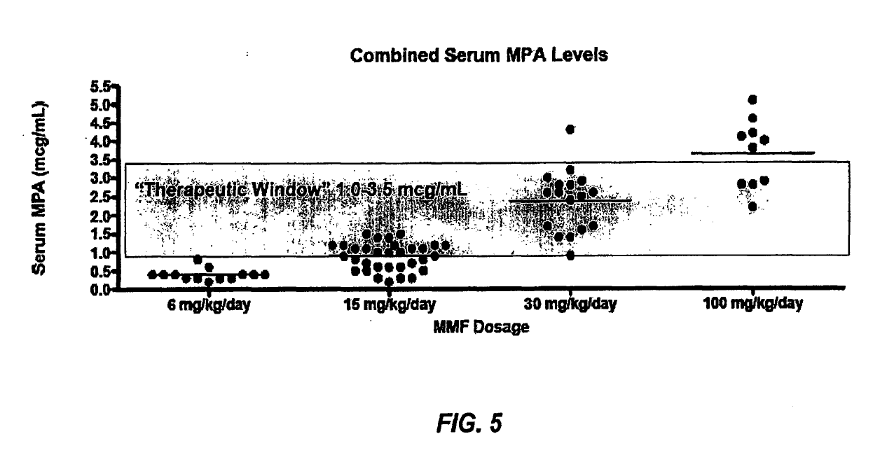

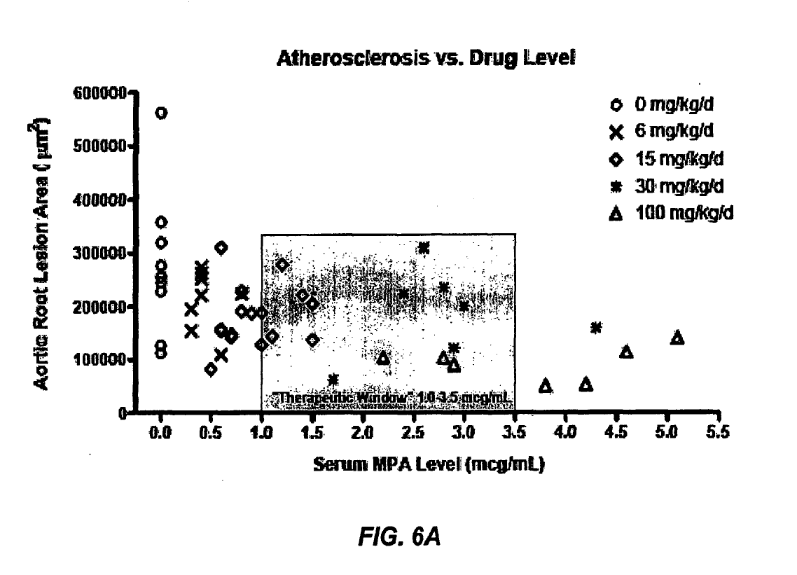

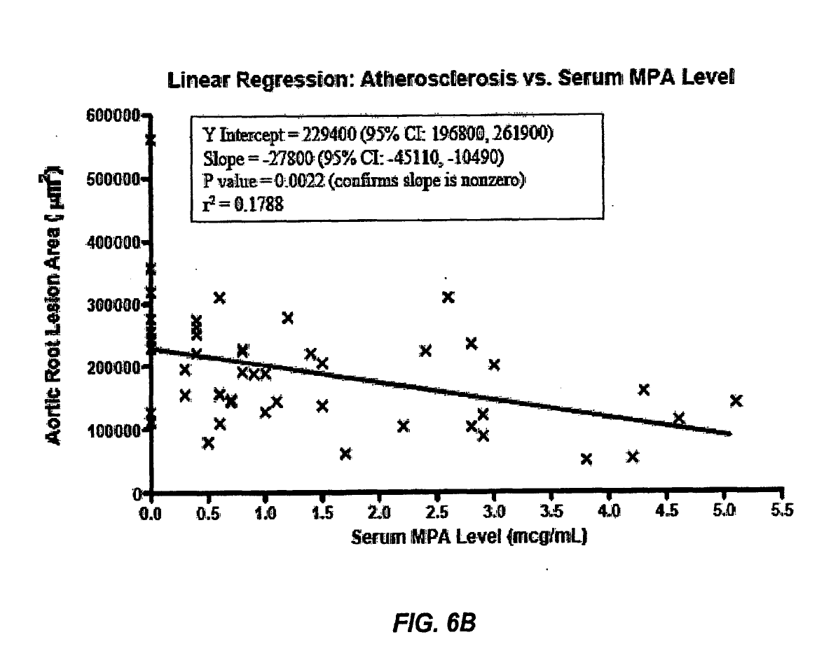

The data presented herein show that MMF can reduce atherosclerosis in Ldlr−/− mice, even at doses lower than used for preventing organ rejection (see FIG. 1). In addition, MMF treatment alone can lower serum triglyceride and phospholipid levels (FIGS. 2 and 3). MMF alone, however, did not lower serum cholesterol levels (FIG. 4).

### 6.3 Example 3

### Effect of Simvastatin, Combined with MMF on a Mouse Model of Atherosclerosis

This example illustrates the effect of low-dose MMF on mice lacking the low-density lipoprotein receptor. B6.129S7-Ldlrtm1Her/J (Ldlr−/− mice) animals were obtained from Jackson Labs and placed on a high-fat, high-cholesterol “Western-type” diet (WTD).

Male, or female animals where indicated, were fed a high-fat, high-cholesterol WTD (15% cocoa butter, 0.25% cholesterol by weight, 34% of calories from fat (“Diet W” Hope Farms, Woerden, Nertherlands)) for 8 or 12 weeks.

As noted above, male Ldlr−/− mice consume about 3.0 to 3.3 grams of Diet W per mouse per day. For treatment, Diet W was formulated with CellCept® incorporated directly into the chow, at concentrations of 0.0064%, 0.0134%, 0.025% or 0.08% mycophenolate mofetil by weight. With this protocol, the mice consume about 6 mg/kg/day, 15 mg/kg/day, 30 mg/kg/day, or 100 mg/kg/day of the MMF, respectively. Where the animals were treated with a statin, the diet was formulated to include MMF as above, and Simvastatin at 0.075% (w/w), such that the mice would consume 90 mg/kg/day of the statin. Mice were sacrificed 8 weeks after feeding on the study diet, and the endpoints determined.

The primary endpoint was evaluated using histological analysis of plaque area at the aortic root. The total lesion area in oil red O-stained cryostat sections of the aortic root was quantified using the Leica image analysis system. Mean lesion area (in square millimetres) was calculated from 4 oil red O-stained sections, beginning at the appearance of the tricuspid valves.

The average atherosclerotic area was compared between the groups using the one-way ANOVA test. Additional secondary endpoints examined were plasma levels of total cholesterol, HDL cholesterol, triglycerides, and phospholipids. In female mice, the percentage of T cells with the NKT phenotype was determined using a fluorescence-activated cell sorter. T cells were harvested from spleens collected at the time of sacrifice, and stained with anti-CD3 FITC and CD tetramer using published methods (Chung et al., 2005, J Immunol. 174:3153-3157). Cells that stained positive for CD3 and also bound CD1d tetramer were scored as a percentage of CD3-positive cells.

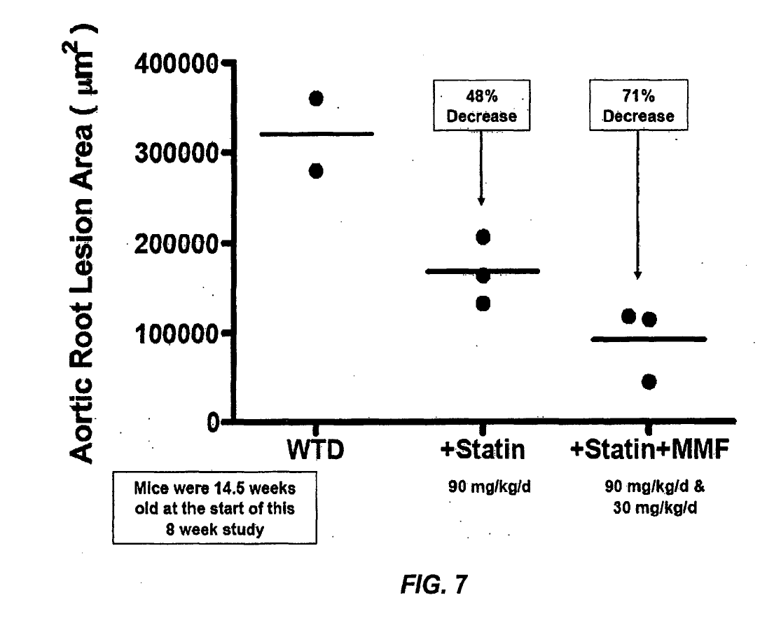

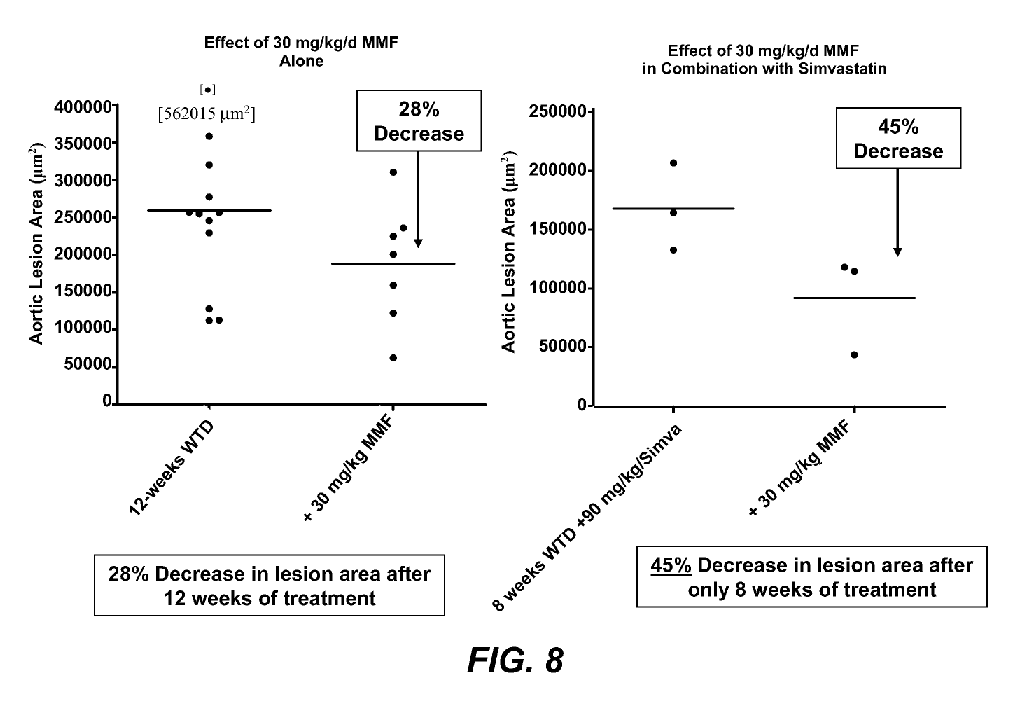

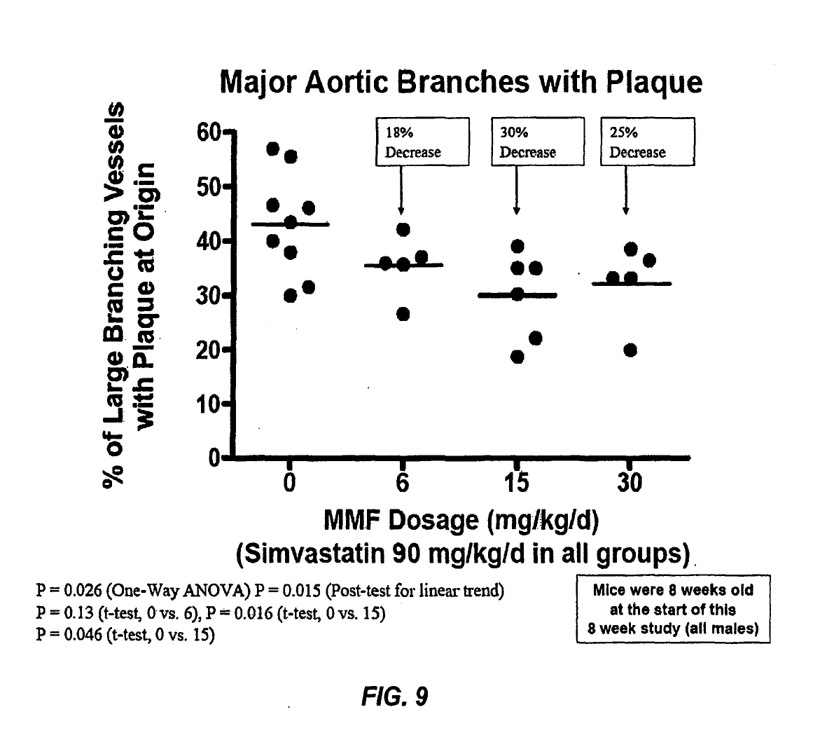

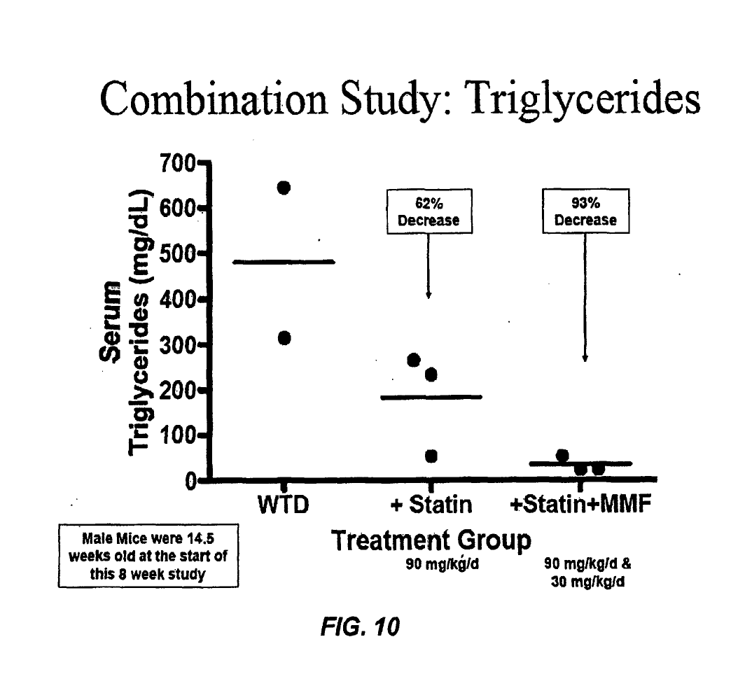

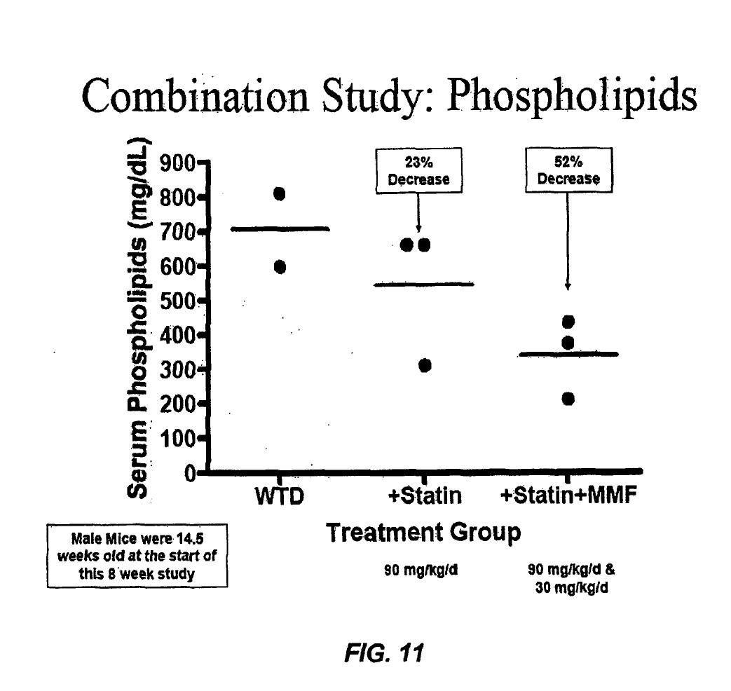

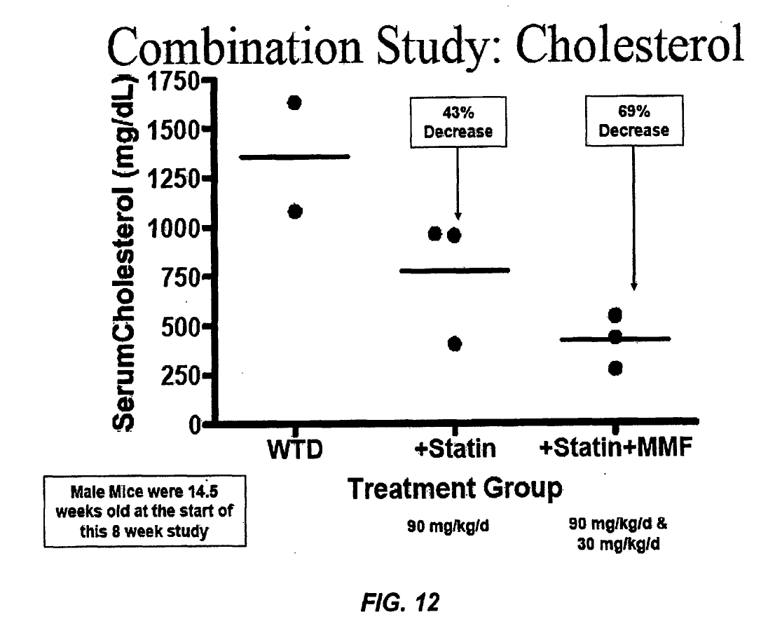

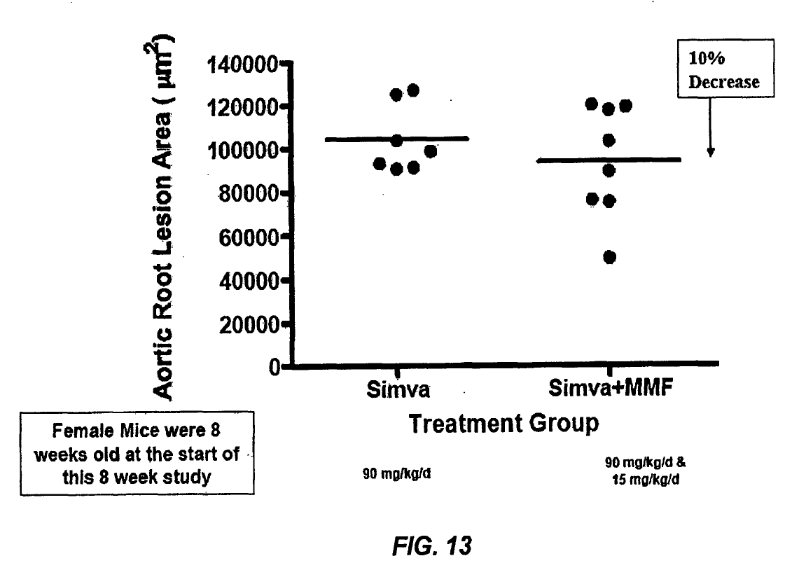

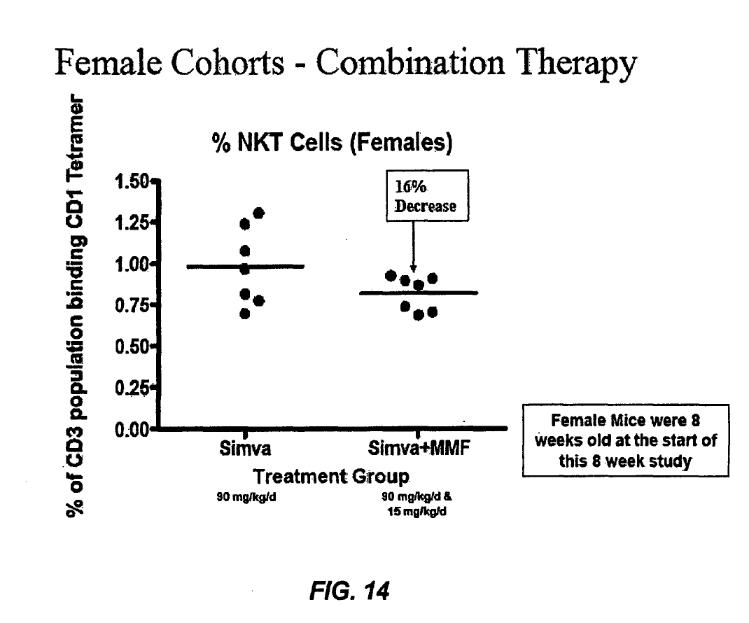

In the present study, the combination of an IMPDH inhibitor and a HMG CoA reductase inhibitor simvastatin showed a reduction of atherosclerotic plaques to levels lower that those observed with the compounds individually (FIGS. 7 and 8: data on left side of FIG. 8 is abstracted from FIG. 1). A reduction in plaque levels were observed in the percentage of major aortic branches, showing plaque was observed in female mice after 8 weeks of combination therapy (FIG. 9). Among younger male mice, a decrease in aortic branch atherosclerosis was also seen, and in older male mice a decrease in lesion area at the aortic root was accompanied by decreases in serum cholesterol, triglyceride, and phospholipid levels to levels lower than those observed with the compounds used alone (FIGS. 7, 10, 11, and 12). The enhanced effect of the combination therapy in reducing plaques appears also to occur in treated female Ldlr−/− mice, a reduction which was accompanied by a decrease in the percentage of natural killer T cells (NKT cells) (FIGS. 13 and 14). In a prior study using older Ldlr−/− male mice, other investigators showed that treatment with simvastatin decreased atherosclerosis but had a minor effect on triglyceride levels (see, e.g., Wang et al., 2002, “Anti-atherosclerotic effect of simvastatin depends on the presence of apolipoprotein E,” Atherosclerosis 162(1): 23-32). The present study suggests that the combination of an IMPDH inhibitor and a statin could be used to lower a risk factor associated with atherosclerosis, an effect not seen in the prior study using statin as a monotherapy.

### 6.4 Example 4

### Effect of Low Dose MMF in High-Cholesterol Rabbit Model

This example illustrates the use of combination MMF and statin therapy in an animal model of atherogenesis.

Study Design: Studies are done in 30 male New Zealand White (NZW) rabbits weighing 1.0 to 1.5 kg at the beginning of the experiment. After obtaining baseline blood samples, rabbits are fed ad libitum a 1% cholesterol diet for 12 weeks. This diet is prepared by dissolving cholesterol (Sigma, St. Louis, Mo.) in 100% ethanol at a temperature of 60° C., mixing this solution with standard rabbit chow (Purina), and allowing the complete evaporation of the ethanol.

The rabbits will be divided into three groups and treated as indicated: (1) CHOL+MMF (extra-low)+simvastatin group (n=10) receives by gastric gavage 5 mg/kg of MMF and 0.5 mg/kg simvastatin in 0.4 ml of water, (2) CHOL+MMF (ultra-low)+atorvastatin group (n=10) receives by gastric gavage 1 mg/kg of MMF and 0.5 mg/kg atorvastatin in 0.4 ml of water; and (3) CHOL group (n=10), which receives by the same route daily 0.4 ml water. Since the MMF is insoluble in water, the drug is individually prepared as a suspension by vigorous shaking immediately before administration. The MMF, statin (i.e., simvastain or atorvastatin) and vehicle are given daily throughout the 12 experimental weeks.

Blood samples for determination of plasma cholesterol and triglyceride are taken every 2 weeks.

A separate set of rabbits of similar weight will be kept for 12 weeks on a standard rabbit chow not supplemented with cholesterol and then sacrificed to determine normal values.

Animals will be sacrificed at the end of the 12th week. The aorta is rapidly dissected and cut from the beginning of the aortic arch to the bifurcation of the iliac vessels. Then, thoracic and abdominal segments of the aorta are separated using the diaphragm as a reference point. Aortic rings of about 1 mm width are cut at the initiation of the aortic arch for histologic and immunohistologic analysis. Then, thoracic and abdominal segments of the aorta are opened longitudinally and photographed for evaluation of the extension of atherosclerotic plaques. The adventitia is then carefully separated and the aortic segments weighed and used for determination of the total cholesterol content.

Determination of aortic cholesterol content: Lipids are isolated from the aortic segments as described by Folch et al., 1957, J Biol Chem. 266:497-509. Briefly, tissue is homogenized in a mixture of chloroform-methanol 2:1 (v:v) in a final volume 20 times the mixture volume. Homogenates are centrifuged at 2500 rpm for 15 min and the supernatant is washed in ionic 0.017% MgCl₂ solution and then centrifuged for 20 min. Lipids are extracted from the lower layer. Cholesterol is determined in the lipid extract by the method of Zlatikis et al., 1953, J. Lab. Clin. Med. 41:486-492.

Expected Results: The size and severity of the atherosclerotic lesion, as measured by the intima/media ratio, and by aortic cholesterol content, are expected to be significantly reduced in the rabbits treated with the MMF/Statin combination therapy. Size and severity of atherosclerotic lesions in rabbits of group (1) are expected to be similar to those of the group (2) group.

### 6.5 Example 5

### Effect of Low-Dose MMF in Combination with Statin on a Mouse Model of SLE

This example will illustrate the effect of the combination therapy on the mouse model of the W/B F1 cross (New Zealand White×BXSB F1), recognized as the closest model to the human disease SLE. WB F1 mice are bred according to previous protocols (Hang et al., 1981, J Exp Med. 154:216-221) and obtained from Jackson Labs. Eighty percent of W/B F1males are expected to develop a degenerative vascular disease confined predominantly to the coronary artery system, which is often associated with myocardial infarction.

Male W/B F1 mice are divided in three groups and treated as indicated: (1) MMF (extra-low) and simvastatin group (n=10) receives by gastric gavage 5 mg/kg of MMF and 0.5 mg/kg simvastatin in 0.1 ml of water; (2) MMF (ultra-low) and atorvastatin group (n=10) receives by gastric gavage 1 mg/kg of MMF and 0.5 mg/kg atorvastatin in 0.1 ml of water; and (3) control group (n=10) which receives by the same route daily 0.1 ml water. Since the MMF is insoluble in water, the drug is individually prepared as a suspension by vigorous shaking immediately before administration. The MMF and statin (i.e., simvastatin or atorvastatin) and vehicle are given daily throughout the 20 experimental weeks.

Survival of control animals is expected to be approximately 50% at the end of 20 weeks. At the end of 20 weeks of treatment, MMF and statin treated animals are expected to demonstrate improved survival and reduced evidence of disease as compared to MMF treatment alone.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of treating cardiovascular disease, comprising administering to a human subject in need thereof mycophenolate mofetil (MMF) or mycophenolic acid (MPA) in combination with a HMG CoA reductase inhibitor effective to treat the cardiovascular disease.

2. The method of claim 1 in which the administered dose is about 250 mg/day to less than about 500 mg/day of MMF, or a corresponding dose of MPA.

3. The method of claim 1 in which the administered dose is about 100 mg/day to less than about 250 mg/day of MMF, or the corresponding dose of MPA.

4. The method of claim 1 in which the administered dose is above about 50 mg/day to less than about 100 mg/day of MMF, or the corresponding dose of MPA.

5. The method of claim 1 in which the administered dose is about 5 mg/day to about 50 mg/day of MMF, or the corresponding dose of MPA.

6. The method of claim 1 in which the cardiovascular disease is peripheral vascular disease.

7. The method of claim 1 in which the cardiovascular disease is atherosclerosis.

8. The method of claim 7 in which the atherosclerosis is other than organ transplant associated atherosclerosis.

9. The method of claim 1 in which the HMG-CoA reductase inhibitor is a statin.

10. The method of claim 9 in which the statin is selected from mevastatin, lovastatin, simvastatin, pravastatin, fluvastatin, atorvastatin, pitavastatin, rosuvastatin and combinations thereof.

11. The method of claim 9 in which the statin is administered at a dose if administered alone is effective to lower serum cholesterol level by 5% or more.

12. The method of claim 9 in which the statin is administered at a dose if administered alone is effective to lower serum cholesterol level by 10%.

13. The method of claim 9 in which the statin is administered at a dose if administered alone is effective to lower serum cholesterol level by up to 20%.

14. The method of claim 9 in which the statin is administered at a dose if administered alone is effective to lower serum cholesterol level by up to 30%.

15. The method of claim 9 in which the statin is administered at a dose if administered alone is effective to lower serum cholesterol level by up to 40%.

16. The method of claim 9 in which the statin is administered at a dose if administered alone is effective to lower serum cholesterol level by up to 60%.

17. The method of claim 9 in which the statin is administered at a dose of about 5 to about 100 mg/day.

18. The method of claim 9 in which the statin is administered at a dose of about 20 to about 80 mg/day.

19. A method of reducing serum cholesterol level, comprising administering to a human subject in need thereof mycophenolate mofetil (MMF) or mycophenolic acid (MPA) in combination with a HMG CoA reductase inhibitor effective to lower serum cholesterol below the level achievable with the HMG CoA reductase inhibitor alone.

20. The method of claim 19 in which the administered dose is about 250 mg/day to less than about 500 mg/day of MMF, or the corresponding dose of MPA.

21. The method of claim 19 in which the administered dose is about 100 mg/day to less than about 250 mg/day of MMF, or the corresponding dose of MPA.

22. The method of claim 19 in which the administered dose is above about 50 mg/day to less than about 100 mg/day of MMF, or the corresponding dose of MPA.

23. The method of claim 19 in which the administered a dose is about 5 mg/day to about 50 mg/day of MMF, or the corresponding dose of MPA.

24. The method of claim 19 in which the HMG-CoA reductase inhibitor is a statin.

25. The method of claim 24 in which the statin is selected from mevastatin, lovastatin, simvastatin, pravastatin, fluvastatin, atorvastatin, pitavastatin, rosuvastatin and combinations thereof.

26. The method of claim 24 in which the statin is administered at a dose if administered alone is effective to lower serum cholesterol level by 5% or more.

27. The method of claim 24 in which the statin is administered at a dose if administered alone is effective to lower serum cholesterol level by up to 10%.

28. The method of claim 24 in which the statin is administered at a dose if administered alone is effective to lower serum cholesterol level by up to 20%.

29. The method of claim 24 in which the statin is administered at a dose if administered alone is effective to lower serum cholesterol level by up to 40%.

30. The method of claim 24 in which the statin is administered at a dose if administered alone is effective to lower serum cholesterol level by up to 50%.

31. The method of claim 24 in which the statin is administered at a dose if administered alone is effective to lower serum cholesterol level by up to about 70%.

32. The method of claim 24 in which the statin is administered at a dose of about 5 to about 100 mg/day.

33. The method of claim 24 in which the statin is administered at a dose of about 20 to about 80 mg/day.

34. The method of claim 19 in which the subject has abnormally elevated serum cholesterol level.

35. The method of claim 34 in which the serum cholesterol level is 200 mg/dL or higher.

36. The method of claim 34 in which the serum cholesterol level is 240 mg/dL or higher.

37. The method of claim 34 in which the serum cholesterol level is 280 mg/dL or higher.

38. A method of treating hypertriglyceridemia, comprising administering to a human subject in need thereof mycophenolate mofetil (MMF) or mycophenolic acid (MPA) in combination with a HMG CoA reductase inhibitor effective to treat the hypertriglyceridemia.

39. The method of claim 38 in which the administered dose is about 250 mg/day to less than about 500 mg/day of MMF, or the corresponding dose of MPA.

40. The method of claim 38 in which the administered dose is about 100 mg/day to less than about 250 mg/day of MMF, or the corresponding dose of MPA.

41. The method of claim 38 in which the administered dose is above about 50 mg/day to less than about 100 mg/day of MMF, or the corresponding dose of MPA.

42. The method of claim 38 in which the subject is administered a dose equivalent to a human dose of about 5 mg/day to about 50 mg/day of MMF, or the corresponding dose of MPA.

43. The method of claim 38 in which the HMG-CoA reductase inhibitor is a statin.

44. The method of claim 43 in which the statin is selected from mevastatin, lovastatin, simvastatin, pravastatin, fluvastatin, atorvastatin, pitavastatin, rosuvastatin and combinations thereof.

45. The method of claim 43 in which the statin is administered at a dose if administered alone is effective to lower serum triglyceride level by 5% or more.

46. The method of claim 43 in which the statin is administered at a dose if administered alone is effective to lower serum triglyceride level by up to 10%.

47. The method of claim 43 in which the statin is administered at a dose if administered alone is effective to lower serum triglyceride level by up to 20%.

48. The method of claim 43 in which the statin is administered at a dose if administered alone is effective to lower serum triglyceride level by up to 30%.

49. The method of claim 43 in which the statin is administered at a dose if administered alone is effective to lower serum triglyceride level by up to 40%.

50. The method of claim 43 in which the statin is administered at a dose if administered alone is effective to lower serum triglyceride level by up to 60%.

51. The method of claim 43 in which the statin is administered at a dose of about 5 to about 100 mg/day.

52. The method of claim 43 in which the statin is administered at a dose of about 20 to about 80 mg/day.

53. The method of claim 38 in which the hypertriglyceridemia treated is associated with a condition selected from the group consisting of obesity, metabolic syndrome (diabesity), insulin resistance, Type 2 diabetes, non-alcoholic fatty liver disease (NAFLD), alcoholic hepatosteatosis, hepatic cirrhosis, gout, hypothyroidism, nephritic syndrome, uremia, hyperuricemia, acute pancreatitis, chronic pancreatitis, obstructive liver disease, malignancy associated dysproteinemia, drug induced hypertriglyceridemia, dialysis associated hypertriglyceridemia, and familial hyperlipidemia.

54. The method of claim 38 in which the hypertriglyceridemia treated is associated with a condition selected from the group consisting of transplant rejection associated hypertriglyceridemia, toxic chemical associated hepatic steatosis, Tangier disease, familial hypoalphalipoproteinemia, glucose-6-phosphate deficiency, glycogen storage disease, familial hypertriglyceridemia, sporadic hypertriglyceridemia, familial hypercholesterolemia, sporadic hypertriglyceridemia, primary hyperinsulinism, leprechaunism, hereditary pancreatitis, lipoprotein lipase deficiency, lipase-1-deficiency, RP1-associated hypertriglyceridemia, lecithin-cholesterol acyltransferase deficiency, familial combined hyperlipidemia, familial partial lipodystrophy, HIV-associated lipodystrophy, acquired partial lipodystrophy, autoimmunity associated lipodystrophy, familial hemophagocytic lymphohistiocytosis, congenital generalized lipodystrophy, insulin resistant diabetes mellitus, acanthosis nigricans, congenital leptin deficiency, Prader Willi Syndrome, Rabson-Mendenhall Syndrome, Alstrom Syndrome, Cohen Syndrome, POMC Deficiency, monogenic obesity syndromes, idiopathic hepatosteatosis, fatty acid oxidation disorder, apolipoprotein C-II deficiency, apolipoprotein C-III deficiency, apolipoprotein E deficiency, apolipoprotein A-V deficiency, abetalipoproteinemia, hyperapobetalipoproteinemia, ataxia-telangiectasia, multiple symmetric lipomatosis, Mediastino-abdominal lipomatosis, erythropoietic-protoporphyria, Alagille syndrome, Sea Blue histiocyte disease, Niemann-Pick disease, cystic fibrosis, Wilson disease, alpha-1-antitrypsin deficiency, CD36 platelet glycoprotein VI deficiency, insulin receptor substrate-1 deficiency, LDHCP (lipoatrophy with diabetes, hepatic steatosis, hypertrophic cardiomyopathy and leukomelanodermic papules), carnitine palmytoyltransferase deficiency, familial hyperchylomicronemia (due to a circulating inhibitor of lipoprotein lipase).

55. A composition comprising mycophenolate mofetil (MMF) of 500 mg or less, or an equivalent dose of mycophenolic acid, and a HMG CoA reductase inhibitor.

56. The composition of claim 55 in which the MMF is about 250 mg to less than about 500 mg, or the equivalent dose of MPA.

57. The composition of claim 55 in which the MMF is about 100 mg to less than about 250 mg, or the equivalent dose of MPA.

58. The composition of claim 55 in which the MMF is above about 50 to less than about 100 mg, or the equivalent dose of MPA.

59. The composition of claim 55 in which the MMF is about 5 to about 50 mg, or the equivalent dose of MPA.

60. The composition of claim 55 in which the HMG CoA reductase inhibitor is a statin.

61. The composition of claim 60 in which the statin is selected from the group consisting of mevastatin, lovastatin, simvastatin, pravastatin, fluvastatin, atorvastatin, rosuvastatin and combinations thereof.

62. The composition of claim 60 in which the statin is present at about 5 to about 100 mg.

63. The composition of claim 60 in which the statin is present at about 20 to about 80 mg.
